(19) United States
(12) Patent Application Publication
(10) Pub. No.: US 2023/0266319 A1
(43) Pub. Date: Aug. 24, 2023

(54) PREDICTING IMMUNOTHERAPY RESPONSE

(71) Applicant: TECHNION RESEARCH & DEVELOPMENT FOUNDATION LIMITED, Haifa (IL)

(72) Inventors: Timothy Jon COOPER, Haifa (IL); Yuval SHAKED, Haifa (IL); Madeleine Rose BENGUIGUI, Haifa (IL)

(21) Appl. No.: 18/140,177

(22) Filed: Apr. 27, 2023

Related U.S. Application Data
(63) Continuation of application No. PCT/IL2021/051278, filed on Oct. 28, 2021.
(60) Provisional application No. 63/106,432, filed on Oct. 28, 2020.
(60) Provisional application No. 63/228,128, filed on Aug. 1, 2021.

(51) Int. Cl. G01N 33/569; A61K 35/17

(52) U.S. Cl. CPC G01N 33/56972; A61K 35/17; G01N 2800/52

(57) ABSTRACT

Methods of determining suitability to be treated with an immunotherapy comprising receiving a sample from the subject and determining the presence of Ly6E expressing neutrophils in the sample, wherein the presence of the Ly6E neutrophils indicates the subject is suitable for treatment are provided. Pharmaceutical composition comprising Ly6E neutrophils, and methods of treatment by administering Ly6E neutrophils are also provided.

### CROSS-REFERENCE TO RELATED APPLICATIONS

This application is a Bypass Continuation Application of PCT Patent Application No. PCT/IL2021/051278 having International filing date of Oct. 28, 2021, which claims the benefit of priority of U.S. Provisional Patent Application Nos. 63/106,432 filed Oct. 28, 2020, and 63/228,128 filed Aug. 1, 2021 the contents of which are all incorporated herein by reference in their entirety.

### FIELD OF INVENTION

The present invention is in the field of immunotherapy.

### BACKGROUND OF THE INVENTION

The discovery of immune checkpoints has led to the development of a new generation of cancer immunotherapies in the form of immune checkpoint inhibitors (ICIs). These agents have revolutionized cancer treatment as the focus of treatment has shifted from the tumor itself to the host's immune system. The first immune checkpoint proteins that were discovered were CTLA-4, PD-1 and its ligand PD-L1. These proteins, expressed by immune cells (CTLA-4, PD-1) and by tumor cells (PD-L1), contribute to the exhaustion of cytotoxic T lymphocytes (CTLs) therefore inhibiting T cell killing effects and enhancing immune evasion of tumor cells. Antibodies blocking these immune checkpoints have been developed and are currently in use in the clinic with some promising and remarkable successes for the treatment of advanced malignancies such as melanoma, non-small cell lung cancer (NSCLC), renal cell carcinoma and some hematological cancers. However, these ICIs show therapeutic benefit only in a small proportion of cancer patients. Despite durable and sustained responses to ICIs, only a small proportion of patients (˜20-30%) respond to this treatment modality, making the majority of patients resistant to such therapies. Several common cancer types such as breast, prostate, and colon cancers have shown very low frequency of response to ICI therapy. Thus, there is a major need to distinguish patients that will benefit from ICI therapy from those that do not.

There are a few known biomarkers used for clinical decision making when using ICI-based immunotherapy or their combination. It has been suggested that markers, such as PD-L1 expression, mutational burden, and mismatch repair deficiency in tumors can predict the patients that will respond to immunotherapy. Other explored biomarkers related to additional immune cells infiltrating tumors, such as immunosuppressive macrophages and myeloid derived suppressor cells, are negative markers for response. However, many of these biomarkers require biopsies from the tumor in order to identify specific immune cell types or the expression of molecules by tumor cells. Therefore, there is a great need to identify systemic biomarkers using liquid biopsies in order to predict therapy response.

Myeloid derived suppressor cells (MDSCs), of which a subcategory are neutrophils, exist at a very low level in the peripheral blood of healthy patients, however, in patients with cancer, the population of MDSCs is substantially expanded. Signals originating from tumors or inflamed tissue stimulate the differentiation of immature myeloid cells into MDSCs and increase their expansion. MDSC expansion is mediated primarily by different chronic inflammatory factors such as granulocyte macrophage-colony stimulating factor (GM-CSF), TNF-α, IL-1β, IL-6, IFN-γ, transforming growth factor beta (TGF-β), and vascular endothelial growth factor beta (VEGF-β). In relation to immunotherapy, it was demonstrated that MDSCs and macrophages contribute to immunotherapy resistance. For example, it was demonstrated that polymorphonuclear (PMN) MDSCs, that infiltrate murine rhabdomyosarcoma, support tumor growth after treatment with anti-PD-1 therapy. The reason for this is that MDSCs suppress immune response and therefore support unresponsiveness to anti-PD-1 therapy in mice. In effect, when PMN-MDSCs were reduced in the tumor, a reduction of the tumor growth was achieved, resulting in a better anti-PD-1 treatment efficacy. In another study, the combination of anti-PD-L1 and a CCR1 inhibitor reduces tumor growth and metastasis. CCR1 promotes the recruitment of MDSCs to the tumor microenvironment and supports their expansion. In the clinic, MDSCs have been shown to correlate with immune suppressive activity in response to ICI therapy. By way of example, in patients with melanoma, the frequency of monocytic MDSCs (M-MDSCs) can be used as a predictive biomarker of response to ipilimumab (anti-CTLA-4). Specifically, it has been shown that lower frequencies of MDSCs in peripheral blood correlate with better treatment outcomes for anti-CTLA-4 therapy. Hence, MDSCs downregulate the immune system and interfere with immunotherapy activity against cancer. Yet, the specific mechanisms by which MDSCs contribute to immunotherapy resistance, and the subset of MDSCs which regulate immunity against cancer have not yet been identified. Taken together, a comprehensive understanding of MDSC biology may yield significantly better methods of predicting response to ICIs—something that is greatly needed.

### SUMMARY OF THE INVENTION

The present invention provides methods of determining suitability to be treated with an immunotherapy, comprising receiving a sample from the subject and determining the presence of Ly6E expressing neutrophils in the sample, wherein the presence of the Ly6E neutrophils indicates the subject is suitable for treatment. Pharmaceutical composition comprising Ly6E neutrophils, and methods of treatment by administering Ly6E neutrophils are also provided.

According to a first aspect, there is provided a method of determining suitability of a subject in need thereof to be treated with an immunotherapy, the method comprising receiving a sample from the subject, and measuring Ly6E expression in neutrophils in the sample, wherein the presence in said sample of neutrophils expressing Ly6E above a predetermined threshold indicates the subject is suitable to be treated with the immunotherapy.

According to another aspect, there is provided a pharmaceutical composition comprising a population of neutrophils expressing Ly6E above a predetermined threshold and a pharmaceutically acceptable carrier, excipient or adjuvant.

According to another aspect, there is provided a method of treating a subject suffering from a disease, the method comprising administering to the subject a pharmaceutical composition of the invention and an immunotherapy, thereby treating the subject.

According to another aspect, there is provided a kit comprising at least one reagent adapted to specifically determine an expression level of Ly6E and at least one reagent adapted to identify a neutrophil.

According to some embodiments, the immunotherapy comprises an immune checkpoint inhibitor (ICI).

According to some embodiments, the ICI comprises at least one of anti-PD-1, anti-PD-L1, anti-PD-L2 and anti-CTLA4 immunotherapy.

According to some embodiments, the subject suffers from cancer.

According to some embodiments, the cancer is selected from lung cancer, breast cancer, colon cancer, and renal cancer.

According to some embodiments, the sample is a sample comprising cells.

According to some embodiments, the sample is a cancer sample, optionally wherein the cancer sample is a tumor biopsy.

According to some embodiments, the sample is a bodily fluid.

According to some embodiments, the bodily fluid is selected from peripheral blood.

According to some embodiments, the sample is acquired from the subject before initiation of administration of the immunotherapy.

According to some embodiments, the method further comprises extracting the sample from the subject.

According to some embodiments, the neutrophils are CD45+, HLA-DR−, Lin−, CD11b+, CD33+, CD14−, and CD15+ cells.

According to some embodiments, the neutrophils are myeloid derived suppressor cells (MDSCs).

According to some embodiments, the MDSCs are granulocytic MDSCs (G-MDSC).

According to some embodiments, the G-MDSC is a polymononuclear (PMN)-MDSC, optionally wherein the PMN-MDSCs are CD45+/CD11B+/Ly6CLow/Ly6G+/Ly6E+ cells.

According to some embodiments, the measuring comprises measuring Ly6E surface protein expression.

According to some embodiments, the measuring comprises flow cytometric analysis.

According to some embodiments, the measuring comprises measuring Ly6E mRNA expression.

According to some embodiments, the measuring comprises single cell RNA analysis, optionally wherein the analysis is RNA sequencing (RNAseq).

According to some embodiments, the neutrophils expressing Ly6E above a predetermined threshold make up greater than a predetermined threshold percentage of all neutrophils in the sample.

According to some embodiments, the predetermined threshold percentage is 70% of neutrophils.

According to some embodiments, the neutrophils expressing Ly6E above a predetermined threshold comprise an mRNA expression profile provided in Table 3.

According to some embodiments, the subject is human, the neutrophils expressing Ly6E above a predetermined threshold are PMN-MDSCs and express at least one of IFIT1, ISG15, IFIH1, HERC5, RSAD2, IFI6, MT2A, EPSTI1, CMPK2, CMTR1, IFI44L, DHX58, SERTM2 and IFNW1.

According to some embodiments, the subject is human, the neutrophils expressing Ly6E above a predetermined threshold are PMN-MDSCs and comprise increased expression of at least one of IFIT3, IFIT1, IFIT2, STAT1, ISG15, STAT2, IFIT5, and IL1B.

According to some embodiments, the method further comprises administering the immunotherapy to the suitable subject or administering the immunotherapy and a pharmaceutical composition of the invention to an unsuitable subject.

According to some embodiments, the composition is formulated for administration to a human subject.

According to some embodiments, the population of neutrophils expressing Ly6E above a predetermined threshold make up at least 40% of all neutrophils in the composition.

According to some embodiments, the disease is a disease suitable to be treated by the immunotherapy.

According to some embodiments, the subject does not respond to or is predicted not to respond to the immunotherapy.

According to some embodiments, the predicting comprises a method of the invention.

According to some embodiments, the immunotherapy comprises immune checkpoint inhibition.

According to some embodiments, the disease is cancer.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: A multi-model approach to identify a clinically relevant biomarker for immunotherapy. A schematic overview of the approach is provided (see Materials and Methods for details).

FIGS. 2A-G: Response to anti-PD1 therapy in various tumor models. (2A-C) Averaged tumor growth profiles for: (2A) BALB/c mice implanted with parental or mutagenized 4T1 breast cancer (4T1P and 4T1M) and treated with αPD1 or control IgG antibodies (n=5 mice/group); (2B) BALB/c mice implanted with spontaneously responding EMT6 breast cancer (IgG n=5, αPD1 n=45); (2C) C57BL/6×CBA backcrossed mice implanted with parental LLC lung cancer (IgG n=5, αPD1 n=15). (2D-F) Raw, individual tumor growth profiles of: (2D) BALB/c mice implanted with parental or mutagenized 4T1 breast cancer (4T1P and 4T1M) and treated with αPD1 or control IgG antibodies (n=5 mice/group); (2E) BALB/c mice implanted with spontaneously responding EMT6 breast cancer (IgG n=5, αPD1 n=45); (2F) C57BL/6×CBA backcrossed mice implanted with parental LLC lung cancer (IgG n=5, αPD1 n=15). For 2B-C and 2E-F, unsupervised, hierarchical clustering and pairwise comparison to respective control, IgG-treated mice was utilized to segregate mice into non-responding (NR) and responding (R) groups. Treatment was initiated at a tumor size of −50 mm3 (arrow). Significance was assessed by means of two-sample KS-test (**=p<0.001, ***=p<0.0001). IgG=control, NR=non-responder, PR=partial responder, R=responder. Treatment was initiated at a tumor size of −50 mm3 (arrow). (2G) Principal component analysis, based on the frequencies of all major immune cell-types (NK, B, CD8/4+ T-cells, monocytes, granulocytes) in the blood of non-tumor bearing C57BL/6 (n=13) and C57BL/6×CBA backcross (n=13) mice, as determined by flow cytometry and expressed as a % of CD45+ cells. 95% confidence zones are shown (ellipses).

FIGS. 3A-H. IFN-stimulated, Ly6Ehi neutrophils mark response to anti-PD1 in 4T1 breast cancer. 10× scRNA-seq was performed on GR1+ cells obtained from parental (P) (non-responsive) and mutagenized (M) (responsive) 4T1 breast cancer tumors. (3A) UMAP plot of 2886 filtered, GR1+ neutrophils (4T1P=681 cells, 4T1M=2185 cells), with cells colored based on differential abundance score. Two significantly enriched, cellular neighborhoods (dotted lines) are highlighted. The top 10, most significant marker genes of each neighborhood are listed (FDR<0.001, log 2 fold-change >1.5). Monocytes (not shown) were discarded from analysis. (3B) Trajectory analysis for 12 distinct, GR1+ granulocytic clusters. Solid black line=trajectory lineages, which form the basis of the pseudotemporal ordering as inferred by partition-graph based abstraction (PAGA). Black arrows=simplified RNA-velocity. (3C) Top: Histogram of binned cell frequencies as a function of aligned pseudotime. Smoothed distributions, generated by loess regression, are overlaid. Significance was assessed by means of two-sample KS-test. Bottom: Heatmap displaying normalized, binned enrichment scores for all gene modules that display a significant association with pseudotime (FDR<0.01). Only gene-modules common to both lineages are shown. (3D) Boxplot showing the levels of IFNγ, TNFα and IFNα within untreated 4T1 tumor lysates. (3E) Binned, normalized expression of Ly6E. Data was imputed for visual clarity. (3F) Frequency of Ly6G+Ly5CloLy6Ehi neutrophils in 4T1 tumors, as determined by flow cytometry. (3G) Gating strategy of Ly6Ehi neutrophils in mouse. (3H) Gating strategy C57BL/6 mice bearing parental (LLCP, left) or mutagenized (LLCM, right) Lewis lung cancer. In 3D and 3F significance was assessed by means of a Mann-Whitney test (**=p<0.001, ***=p<0.0001).

FIGS. 4A-H: Flow cytometry validation of Ly6E(hi) Neutrophils in responsive and non-responsive cell lines. (4A-C) Frequency of Ly6G+Ly6C(lo)Ly6E(hi) neutrophils, as determined by flow cytometry, in the blood of (4A) BALB/c mice bearing parental (P) and mutagenized (M) 4T1 breast tumors; (4B) BALB/c mice bearing responsive (R) and non-responsive (NR) EMT6 breast tumors; and (4C) C57BL/6×CBA backcrossed mice bearing parental LLC lung cancer. Tumor growth for all individual mice profiled is shown in FIG. 2D-F, respectively. (4D) Frequency of Ly6G+Ly6C(lo)Ly6E(hi) neutrophils as a percentage of all neutrophils, as determined by flow cytometry, in the blood of BALB/c mice bearing parental (P) and mutagenized (M) 4T1 breast tumors just before administration of anti-PD-L1 therapy, anti-CTLA4 therapy or control IgG. (4E-F) Averaged tumor growth profiles for: BALB/c mice implanted with parental or mutagenized 4T1 breast cancer (4T1P and 4T1M) and treated with (4E) αPD-L1 or control IgG antibodies or (4F) αCTLA4 or control IgG antibodies. (4G-H) Tumor growth for the individual mice used in (4G) 4E and (4H) 4F are shown. All blood samples were taken at baseline at an average tumor size of ˜50 mm\hat{ }3. Significance was assessed by means of a Mann-Whitney test (***=p<0.0001). Grey arrows indicate the commencing of ICI treatment when the average tumor size reached ˜50 mm\hat{ }3.

FIGS. 5A-E: Functional characterization of Ly6E(hi) neutrophils. (5A) Schematic of adoptive transfer. Isolated GR1+ cells are treated in-vitro with IFNα/γ, inducing a Ly6Ehi-like state characterized by secretion of effector molecules, and injected into BALB/c mice bearing parental, non-responsive 4T1 breast tumors. (5B) Frequency of Ly6G+Ly5CloLy6Ehi neutrophils following exposure of GR1+ cells to IFNγ, IFNα or both, as determined by flow cytometry. Significance was assessed by means of a Mann-Whitney test (***=p<0.0001). (5C) Heatmap comparing normalized, log 2-fold changes from RT-qPCR (treated [+IFNγ/α] vs. un-treated [CTRL] GR1+ cells) and scRNA-seq (Ly6Ehi neutrophils vs. all remaining neutrophils). SC=scRNA-seq. μ=averaged RT-qPCR values. (5D) Averaged tumor growth profiles for mice bearing parental, non-responsive 4T1 breast tumors treated with either a monotherapy (control IgG or αPD1) or a combined therapy, with untreated GR1+ or treated GR1+Ly6Ehi cells, as specified. (5E) Raw, individual tumor growth profiles of mice bearing parental, non-responsive 4T1 breast tumors treated with either a monotherapy (control IgG or αPD1) or a combined therapy, with un-treated GR1+ or treated GR1+Ly6Ehi cells, as specified. Treatment was initiated at a tumor size of −50 mm3 (arrow).

FIGS. 6A-B: An IFN-response signature in murine Ly6E+ Neutrophils. (6A) Over-representation analysis was performed on 348 genes whose expression levels are increased >=1.5-fold in Ly6E(hi) neutrophils (relative to all other neutrophils). All significant results are presented (FDR p<0.01, permutations test). GeneRatio=percentage of genes in each category. (6B) Visualization of key interferon (IFN)-stimulated gene expression in all murine neutrophils. Note the overlap in expression with Ly6E (top left).

FIGS. 7A-B: Human cells equivalent to murine Ly6E(hi) neutrophils. (7A) Human granulocytes and neutrophils (PMN-MDSCs) were isolated from scRNA-seq data of 7 non-small-cell lung cancer (NSCLC) patients (PMID: 30979687) (blood). Data was processed, clustered and subject to over-representation analysis in an identical manner to that of the murine data shown in FIG. 6A. All significant results are presented (FDR p<0.01, permutations test). GeneRatio=percentage of genes in each category. (7B) Visualization of key interferon (IFN)-stimulated gene expression in all human granulocytes/neutrophils/PMN-MDSCs. Note the same genes are shown as in FIG. 6B.

FIGS. 8A-G: Ly6Ehi neutrophils as a biomarker for immunotherapy in humans. (8A) UMAP plot of 11702 filtered, CD45+ cells taken from publicly available non-small cell lung cancer (NSCLC) scRNA-seq data (patient blood samples at baseline, n=8), with cells colored by cell type. (8B) Binned UMAP plot of isolated neutrophils (dotted box in 8A), with cells colored by the extent of enrichment for a Ly6Ehi functional signature. The top 10, most significant marker genes of the enriched cluster (dotted lines) are listed (FDR<0.001, log 2 fold-change >1.5). (8C) Binned, normalized expression of Ly6E. Data was imputed for visual clarity. (8D-E) Frequency of CD14-CD15+LY6E(hi) neutrophils in the blood of (8D) an independent cohort of NSCLC patients and (8E) an independent cohort of melanoma patients, as determined by flow cytometry. Data is stratified by response rate based on RECIST category at 3 months (PD=progressive disease, SD=stable disease, PR=partial response, CR=complete response). Sample sizes are denoted for each group. Significance was assessed by means of a Mann-Whitney test (***=p<0.0001, **=p<0.001, *=p<0.01). (8F) Smoothed area under the curve (AUC)-receiver operating characteristics (ROC) plots for: Ly6E(hi) neutrophils in NSCLC (95% CIs: 0.9287-0.9893), absolute neutrophil count in NSCLC (95% CIs: 0.5016-0.9322), tumor PD-L1 immunohistochemistry (IHC) in NSCLC (95% CIs: 0.354-0.9662) and Ly6E(hi) neutrophils in melanoma (95% CIs: 0.9244-1.0). (8G) Gating strategy for human Ly6E(hi) neutrophils.

### DETAILED DESCRIPTION OF THE INVENTION

The present invention, in some embodiments, provides methods of determining suitability to be treated with an immunotherapy, or method of predicting response to an immunotherapy. Pharmaceutical composition comprising Ly6E positive neutrophils, and methods of treatment by administering the pharmaceutical composition are also provided.

The invention is based on the surprising finding that the presence of neutrophils that highly express Lymphocyte antigen 6E (Ly6E) in subjects suffering from cancer is predictive of the subject being a responder to immunotherapy. Ly6E expression has previously been reported to correlate with poor prognosis and poor overall survival in cancer subjects. Further, MDSCs highly expressing Ly6E are informative. MDSCs are generally immunosuppressive, and their presence is thought to inhibit immunotherapy. Thus, it is highly surprising that the presence of this specific cell population, both in the tumor and in peripheral blood, should prognose a positive response to immunotherapy.

Prognosis

By a first aspect, there is provided a method of determining suitability of a subject to be treated with an immunotherapy, the method comprising providing a sample from the subject and determining the presence of lymphocyte antigen 6E (Ly6E) expressing cells in a sample, wherein the presence of the Ly6E expressing cells indicates the subject is suitable to be treated by the immunotherapy.

In some embodiments, the subject is a mammal. In some embodiments, the subject is a human. In some embodiments, the subject is a subject in need thereof. In some embodiments, the subject suffers from a disease. In some embodiments, the subject has been diagnosed with the disease. In some embodiments, the disease is a disease treatable by immunotherapy. In some embodiments, the disease is cancer. In some embodiments, the disease is a proliferative disease. In some embodiments, the proliferative disease is cancer.

In some embodiments, the cancer is a PD-L1 positive cancer. In some embodiments, the cancer is a CTLA-4 positive cancer. In some embodiments, the cancer is a solid cancer. In some embodiments, the cancer is a tumor. In some embodiments, the cancer is a hematopoietic cancer. In some embodiments, cancer is selected from breast cancer, cervical cancer, endocervical cancer, colon cancer, lymphoma, esophageal cancer, brain cancer, head and neck cancer, renal cancer, meningeal cancer, glioma, glioblastoma, Langerhans cell cancer, lung cancer, mesothelioma, ovarian cancer, pancreatic cancer, neuroendocrine cancer, prostate cancer, skin cancer, stomach cancer, tenosynovial cancer, tongue cancer, thyroid cancer, uterine cancer, and testicular cancer. In some embodiments, the cancer is selected from lung cancer, breast cancer, colon cancer, skin cancer and renal cancer. In some embodiments, the cancer is selected from lung cancer, breast cancer, skin cancer and renal cancer. In some embodiments, the cancer is breast cancer. In some embodiments, the cancer is colon cancer. In some embodiments, the cancer is renal cancer. In some embodiments, the cancer is lung cancer. In some embodiments, the cancer is skin cancer. In some embodiments, the lung cancer is non-small cell lung cancer (NSCLC). In some embodiments, the skin cancer is melanoma. In some embodiments, the cancer is squamous cell carcinoma. In some embodiments, the cancer is small cell carcinoma. In some embodiments, the cancer is carcinoma. In some embodiments, the cancer is adenocarcinoma.

In some embodiments, determining suitability comprises determining response to the immunotherapy. In some embodiments, determining suitability comprises determining if the subject is a responder to the immunotherapy. In some embodiments, determining suitability comprises determining if the subject is a non-responder. In some embodiments, the method further comprises treating a subject suitable to receive the immunotherapy with the immunotherapy. In some embodiments, the method further comprises administering the immunotherapy to a subject determined to be suitable. In some embodiments, a subject determined to be suitable is a responder. In some embodiments, a responder is a subject likely to respond.

In some embodiments, the method is a diagnostic method. In some embodiments, the method is a prognostic method. In some embodiments, the method is an in vitro method. In some embodiments, the method is an ex vivo method. In some embodiments, the method is for determining response to immunotherapy. In some embodiments, the method is for determining if a subject is a responder to the immunotherapy. In some embodiments, the method is for determining if a subject is a non-responder to the immunotherapy. In some embodiments, the method is for predicting a subject's response to an immunotherapy. In some embodiments, the method is for monitoring response to the immunotherapy. In some embodiments, the method is for determining if the immunotherapy should continue.

In some embodiments, the immunotherapy is a plurality of immunotherapies. In some embodiments, the immunotherapy is immune checkpoint blockade. In some embodiments, the immunotherapy is immune checkpoint protein inhibition. In some embodiments, immune checkpoint blockade and/or immune checkpoint inhibition comprises administering to the subject an immune checkpoint inhibitor.

As used herein, the term “an immune checkpoint inhibitor (ICI)” refers to a single ICI, a combination of ICIs and a combination of an ICI with another cancer therapy. The ICI may be a monoclonal antibody, a humanized antibody, a fully human antibody, a fusion protein or a combination thereof.

In some embodiments, the immune checkpoint protein is selected from PD-1 (Programmed Death-1) PD-L1 (Programmed Death-ligand 1), PD-L2; CTLA-4 (Cytotoxic T-Lymphocyte-Associated protein 4); A2AR (Adenosine A2A receptor), also known as ADORA2A; BT-H3, also called CD276; BT-H4, also called VTCN1; BT-H5; BTLA (B and T Lymphocyte Attenuator), also called CD272; IDO (Indoleamine 2,3-dioxygenase); KIR (Killer-cell Immunoglobulin-like Receptor); LAG-3 (Lymphocyte Activation Gene-3); TDO (Tryptophan 2,3-dioxygenase); TIM-3 (T-cell Immunoglobulin domain and Mucin domain 3); VISTA (V-domain Ig suppressor of T cell activation). In some embodiments, the immune checkpoint protein is selected from PD-1, PD-L1, PD-L2 and CTLA4. In some embodiments, the immune checkpoint protein is at least one of PD-1, PD-L1, PD-L2 and CTLA4. In some embodiments, the immune checkpoint protein is selected from PD-1, PD-L1/2 and CTLA4. In some embodiments, the immune checkpoint protein is at least one of PD-1, PD-L1/2 and CTLA4. In some embodiments, the immune checkpoint protein is selected from PD-1, PD-L1, and CTLA4. In some embodiments, the immune checkpoint protein is at least one of PD-1, PD-L1, and CTLA4. In some embodiments, the immune checkpoint protein is selected from PD-1, PD-L1 and PD-L2. In some embodiments, the immune checkpoint protein is selected from PD-1 and PD-L1. In some embodiments, the immune checkpoint protein is PD-1. In some embodiments, immune checkpoint blockade comprises an anti-PD-1/PD-L1/PD-L2 immunotherapy. In some embodiments, immune checkpoint blockade comprises an anti-PD-1 immunotherapy. In some embodiments, immune checkpoint blockade comprises an anti-PD-1 and/or anti-PD-L1 immunotherapy. In some embodiments, the immunotherapy is a blocking antibody. In some embodiments, the immunotherapy is administration of a blocking antibody to the subject. In some embodiments, the immune checkpoint protein is CTLA-4.

In some embodiments, the ICI is an antibody. In some embodiments, antibody is a monoclonal antibody (mAb). In some embodiments, the ICI is a mAb against PD-1 or PD-L1. In some embodiments, the ICI is a mAb against PD-1. In some embodiments, the ICI is a mAb against PD-L1. In some embodiments, the ICI is a mAb that neutralizes/blocks the PD-1 pathway. In some embodiments, the ICI is a mAb against PD-1. In some embodiments, the anti-PD-1 mAb is Pembrolizumab (Keytruda; formerly called lambrolizumab. In some embodiments, the anti-PD-1 mAb is Nivolumab (Opdivo). In some embodiments, the anti-PD-1 mAb is Pidilizumab (CT0011). In some embodiments, the anti-PD-1 mAb is any one of REGN2810, AMP-224, MEDI0680, or PDR001. In some embodiments, the ICI is a mAb against PD-L1. In some embodiments, the anti-PD-L1 mAb is selected from Atezolizumab (Tecentriq), Avelumab (Bavencio), and Durvalumab (Imfinzi). In some embodiments, the anti-PD-L1 mAb is selected from Atezolizumab (Tecentriq), and Durvalumab (Imfinzi). In some embodiments, the ICI is a mAb against CTLA-4. In some embodiments, the anti-CTLA4 antibody is Ipilimumab (Yervoy).

In some embodiments, the immunotherapy is administered in combination with one or more conventional cancer therapy including chemotherapy, targeted cancer therapy, steroids and radiotherapy. Combinations of ICI and radiation therapy have been studied in multiple clinical trials. It will be understood by a skilled artisan that the predictive proteins disclosed herein are predictive in immunotherapy as a monotherapy, as well as part of a combination therapy. In some embodiments, the conventional therapy is a chemotherapy. In some embodiments, the chemotherapy is Cisplatin. In some embodiments, the chemotherapy is Carboplatin. In some embodiments, the conventional therapy is an antineoplastic therapy. In some embodiments, the antineoplastic is Alimta.

In some embodiments, the method comprises receiving a sample. In some embodiments, the method comprises obtaining a sample. In some embodiments, the method comprises providing a sample. In some embodiments, the sample is from the subject. In some embodiments, the method further comprises extracting a sample from the subject. In some embodiments, the sample is from before initiation of an immunotherapy in the subject. In some embodiments, an immunotherapy is the immunotherapy. In some embodiments, the sample was acquired from the subject before initiation of an immunotherapy. In some embodiments, the sample comprises cells. In some embodiments, the method comprises isolating cells from the sample. In some embodiments, the method comprises purifying cells from the sample. Methods of cell isolation and purification are well known in the art and include for example, centrifugation, SEPAX and Ficoll gradient separation to name but a few. Any method of isolation or purification may be employed. In some embodiments, the method comprises dissociating cells in the sample. In some embodiments, the method comprises producing a single cell suspension of cells from the sample.

In some embodiments, the sample is a biological sample. In some embodiments, the sample is a fluid. In some embodiments, the fluid is a biological fluid. In some embodiments, the sample is from the subject. In some embodiments, the sample is not a tumor sample. In some embodiments, the sample is a tumor sample. In some embodiments, the sample is not a hematopoietic cancer, and the sample is a blood sample. In some embodiments, the sample is a blood sample. In some embodiments, the sample is a sample that does not comprise cancer cells. In some embodiments, a blood sample is selected from a whole blood sample, a serum sample and a plasma sample. In some embodiments, a blood sample is selected from a whole blood sample, and a plasma sample. In some embodiments, the sample is a plasma sample. In some embodiments, the sample is a whole blood sample. In some embodiments, the sample is a peripheral blood sample. In some embodiments, the biological fluid is selected from, blood, plasma, lymph, cerebral spinal fluid, urine, feces, semen, tumor fluid and gastric fluid.

In some embodiments, the method comprises determining the presence of Ly6E expressing cells in the sample. In some embodiments, an Ly6E expressing cell is an Ly6E positive cell. In some embodiments, determining is detecting. Ly6E is also known as RIG-E, RIGE, SCA-2, TSA-1, lymphocyte antigen 6 complex, locus E and lymphocyte antigen 6 family member E. The sequence of the human Ly6E gene is provided an Entrez gene number 4061 and the mouse is available at 17069. The protein sequence of the human LY6E protein is available in Uniprot entry Q16553 and the mouse is available in entry Q64253. There are two known splice isoforms of human Ly6E, and they are provided in NM_002346 and NM_001127213. These mRNA produce to protein variants of LY6E available in NP_001120685 and NP_002337. There are seven known mouse splice isoforms that lead to seven protein variants. The mRNAs are available in NM_001164036, NM_001164037, NM_001164038, NM_001164039, NM_001164040, NM_008529 and NM_001374138. The protein sequences are available in NP_001157508, NP_001157509, NP_001157510, NP_001157511, NP_001157512, NP_032555, and NP_001361067. Ly6E expression has been implicated in cancer diagnosis and it has been reported to correlate with poor prognosis.

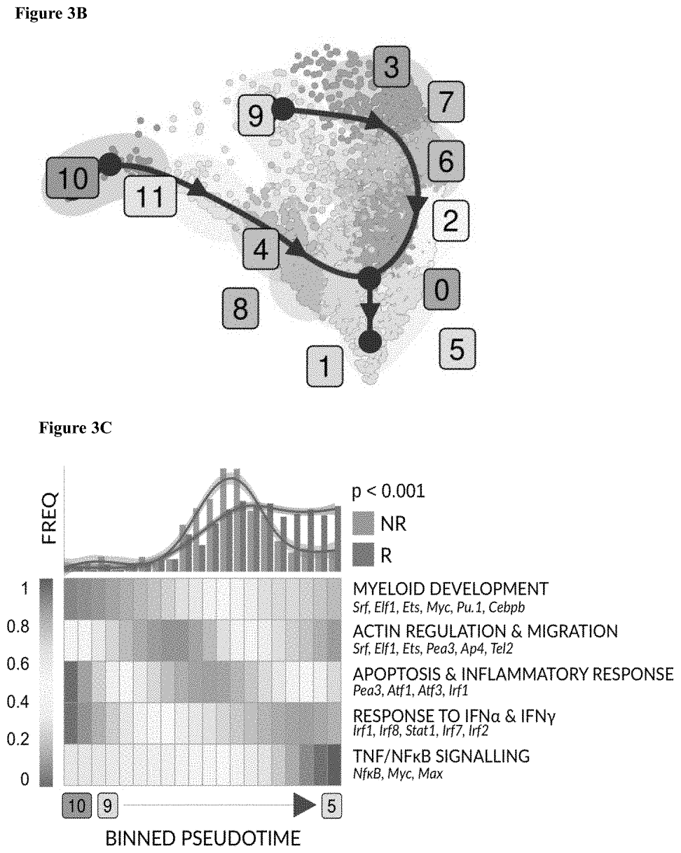

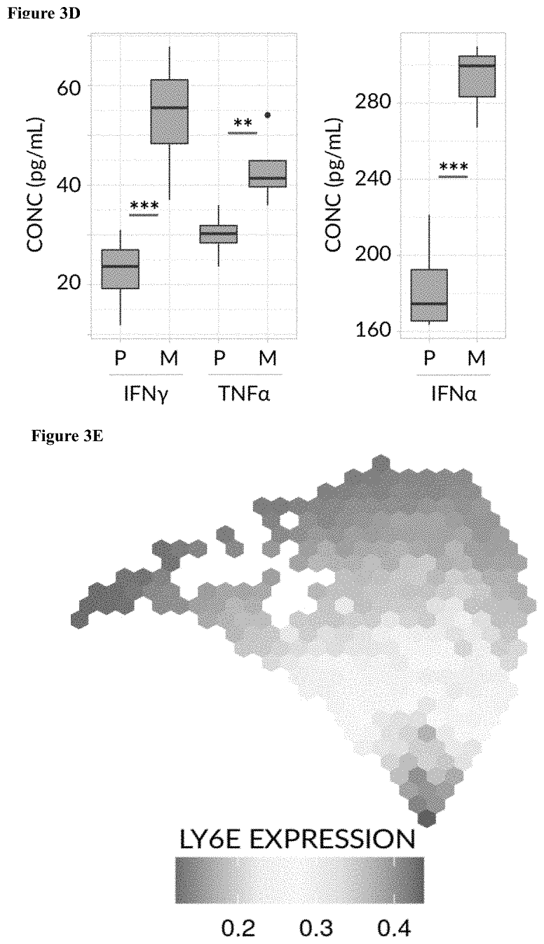

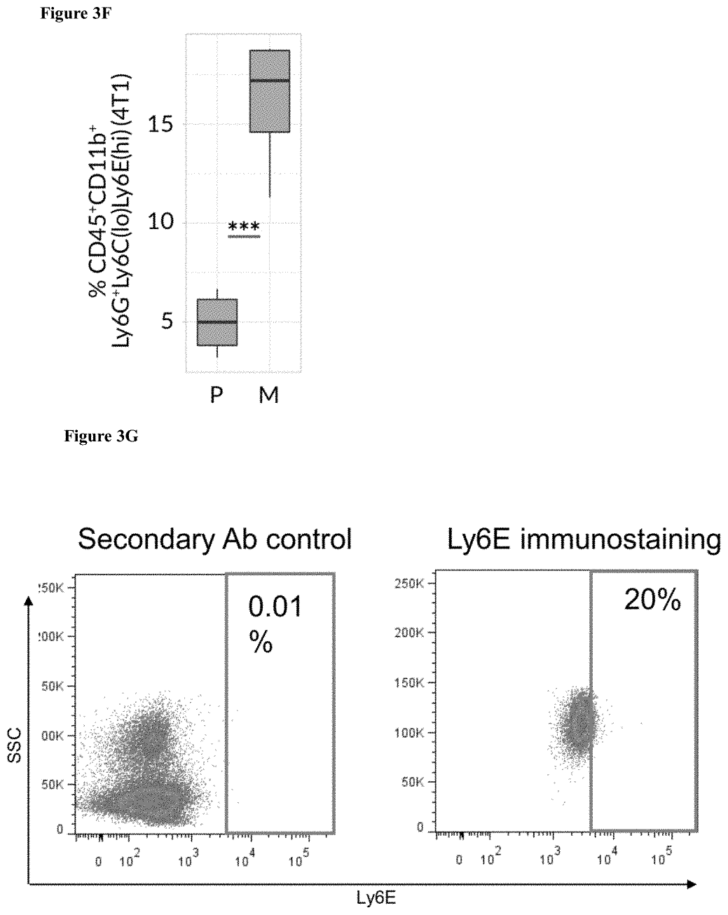

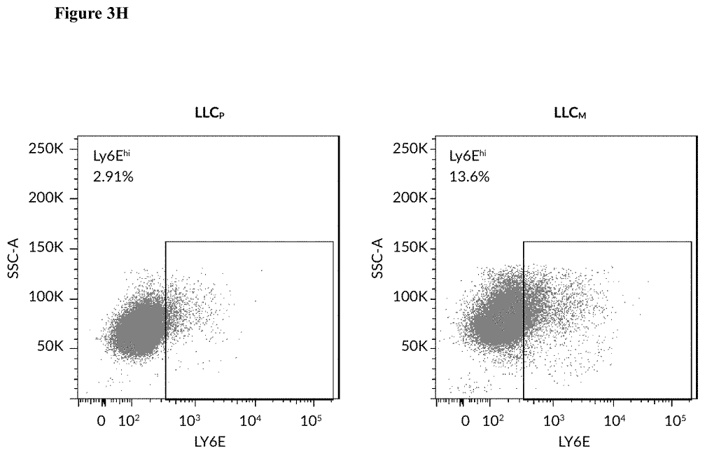

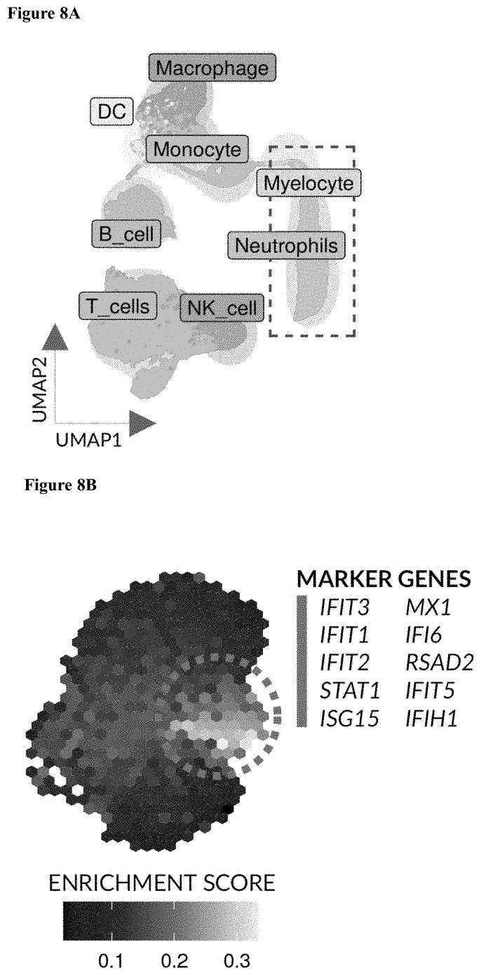

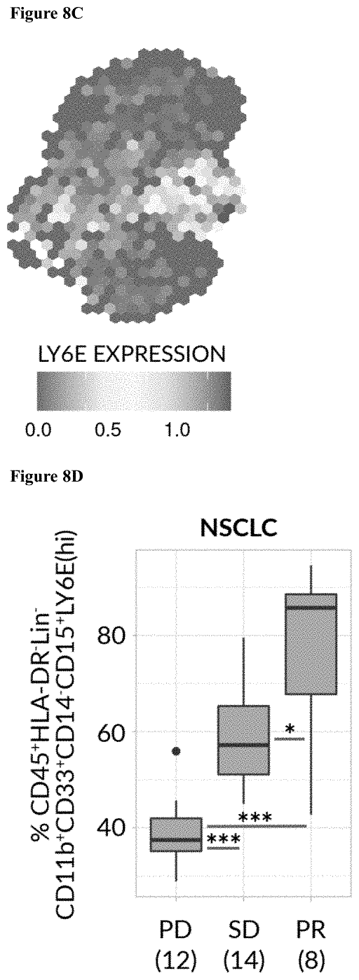

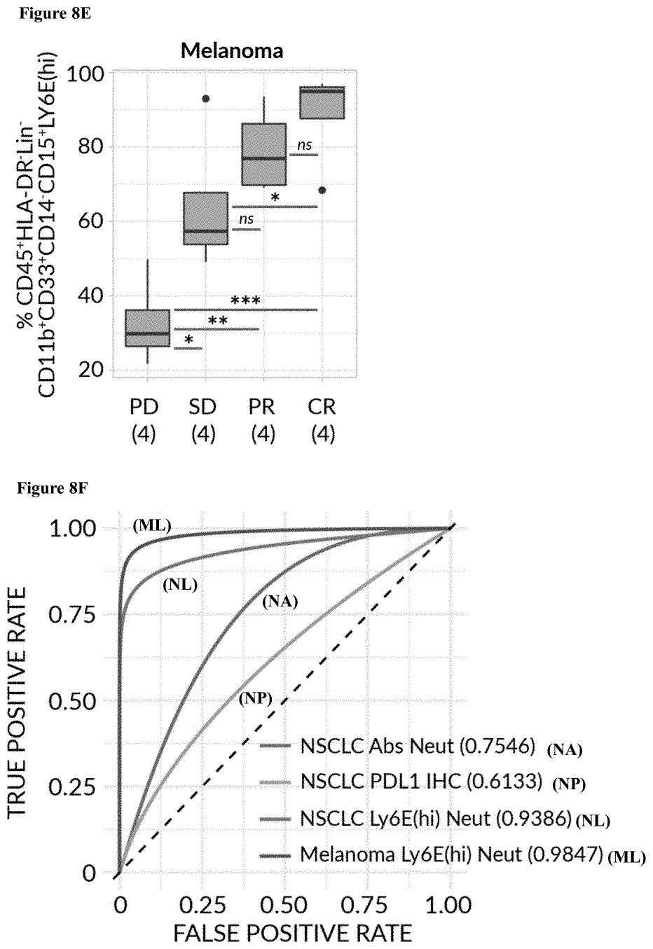

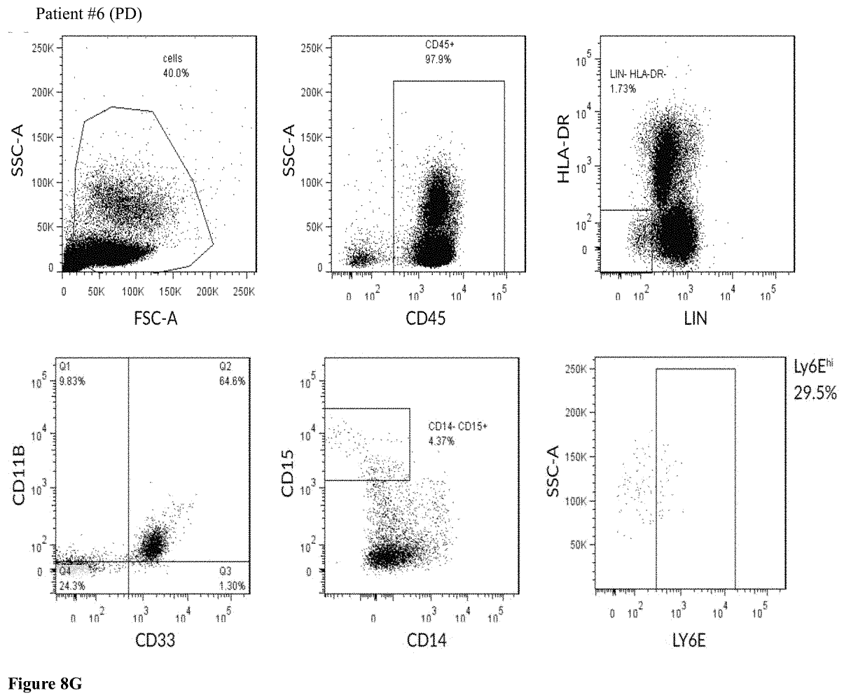

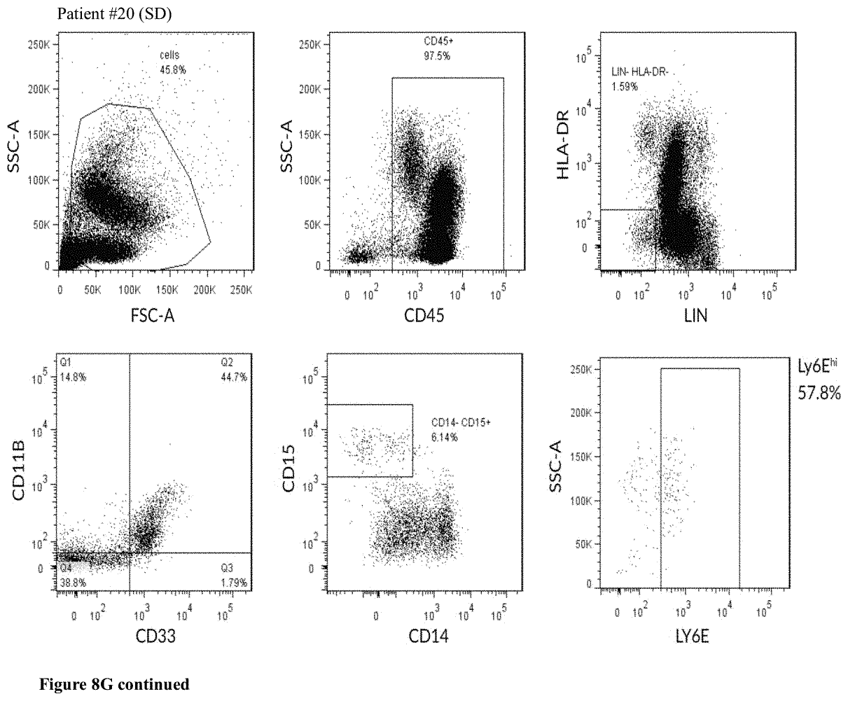

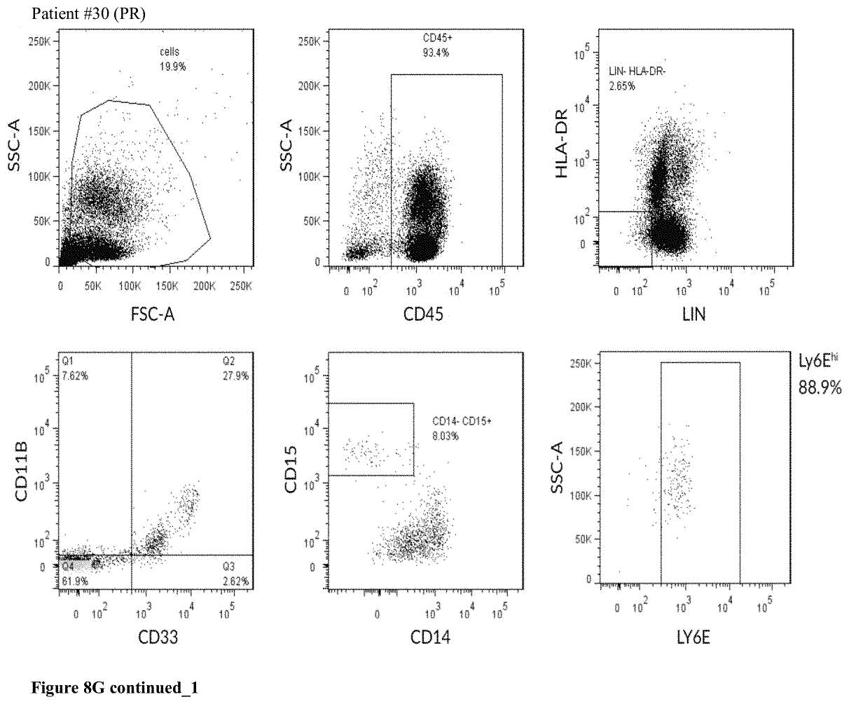

In some embodiments, determining is measuring. In some embodiments, the method comprises measuring Ly6E expression in the sample. In some embodiments, the method comprises measuring Ly6E expression in neutrophils. In some embodiments, measuring comprises determining the presence of neutrophils highly expressing Ly6E. In some embodiments, highly expressing is expressing above a predetermined threshold. Methods of determining proper threshold expression such as during Flow cytometric analysis are well known in the art and examples enabling a skilled artisan to determine such a threshold are provided hereinbelow, such as in FIGS. 3G, 3H and 8G. In some embodiments, measuring is measuring the number of neutrophils with Ly6E expression above the predetermined threshold. In some embodiments, measuring is measuring the percentage of neutrophils in the sample that express Ly6E above the predetermined threshold.

In some embodiments, the presence in the sample of neutrophils expressing Ly6E above the predetermined threshold indicates the subject is suitable to be treated. In some embodiments, the presence in the sample of a population of neutrophils expressing Ly6E above a predetermined threshold indicates the subject is suitable to be treated. In some embodiments, population makes up greater than a predetermined threshold percentage of all neutrophils in the sample. In some embodiments, the presence of highly Ly6E expressing neutrophils making up greater than a predetermined threshold percentage of all neutrophils in the sample indicates the subject is suitable to be treated. In some embodiments, the presence of neutrophils expressing Ly6E above a predetermined threshold making up greater than a predetermined threshold percentage of all neutrophils in the sample indicates the subject is suitable to be treated.

In some embodiments, the threshold percentage is the threshold at which a subject responds to the immunotherapy. In some embodiments, the threshold percentage is the threshold at which the immunotherapy will produce stable disease or response. In some embodiments, the threshold percentage is the threshold at which the immunotherapy will produce response. In some embodiments, the response is partial response. In some embodiments, the threshold percentage is 30%. In some embodiments, the threshold percentage is 40%. In some embodiments, the threshold percentage is 42%. In some embodiments, the threshold percentage is 67%. In some embodiments, the threshold percentage is 70%. In some embodiments, the threshold percentage is 80%. In some embodiments, the threshold percentage is 84%.

In some embodiments, expressing is mRNA expressing. In some embodiments, expressing is protein expressing. In some embodiments, protein expression is surface protein expression. Methods of mRNA detection and measurement are well known in the art and any such method may be employed. These methods include, but are not limited to, PCR, real-time PCR, quantitative PCR, microarray, northern blotting, RNA in-situ hybridization, single cell PCR, sequencing, next-generation sequencing, single cell sequencing and FISH. Methods of protein detection and measurement are also well known in the art and any such method may be employed. These methods include, but are not limited to, western blotting, immunostaining, ELISA, immunohistochemistry, flow cytometry, FACS, protein arrays and antibody-based cell isolation. In some embodiments, the determining comprises flow cytometry. In some embodiments, the measuring comprises flow cytometry. In some embodiments, flow cytometry is flow cytometric analysis. In some embodiments, the determining comprises FACS. In some embodiments, the measuring comprises FACS. In some embodiments, the determining comprises single cell RNA analysis. In some embodiments, the measuring comprises single cell RNA analysis. In some embodiments, the RNA analysis is RNA sequencing (RNA-Seq).

Neutrophils are the most abundant white blood cell found in the body and are well known in the art. Methods of identifying neutrophils and isolating neutrophils are also well known. In some embodiments, the neutrophils are leukocytes. In some embodiments, neutrophils are CD45 positive (CD45+). In some embodiments, neutrophils are HLA-DR negative (HLA-DR−). In some embodiments, neutrophils are lineage negative (Lin−). In some embodiments, neutrophils are CD11B positive (CD11b+). In some embodiments, neutrophils are CD33 positive (CD33+). In some embodiments, neutrophils are CD15 positive (CD15+). In some embodiments, neutrophils are CD14 negative (CD14−). In some embodiments, neutrophils are CD45+, HLA-DR−, Lin−, CD11b+, CD33+, CD14−, and CD15+. In some embodiments, the cells are CD45+, HLA-DR−, Lin−, CD11b+, CD33+, CD14−, and CD15+ cells. In some embodiments, neutrophils are identified as shown in FIG. 8G. In some embodiments, the cell of the invention is CD45+, HLA-DR−, Lin−, CD11b+, CD33+, CD14−, CD15+ and LY6E+. In some embodiments, the cell of the invention is CD45+, HLA-DR−, Lin−, CD11b+, CD33+, CD14−, CD15+ and LY6E(hi).

In some embodiments, the neutrophils are neutrophil-like cells. In some embodiments, a neutrophil-like cell is a cell that is CD45+/CD11b+/Ly6C^{Low}/Ly6G+. In some embodiments, a neutrophil-like cell is a cell that is CD45+, HLA-DR−, Lin−, CD11b+, CD33+, CD14−, and CD15+. In some embodiments, the neutrophils are myeloid derived suppressor cells. In some embodiments, the cell is a myeloid derived suppressor cell (MDSC). In some embodiments, the MDSC is a granulocytic MDSC (G-MDSC). In some embodiments, the MDSC is a monocytic MDSC (M-MDSC). In some embodiments, the G-MDSC is a polymorphonuclear MDSC (PMN-MDSC). In some embodiments, a PMN-MDSC is CD45+/CD11b+/Ly6C^{Low}/Ly6G+. In some embodiments, a PMN-MDSC is identified by its surface protein expression profile. In some embodiments, PMN-MDSC profile comprises CD45+/CD11b+/Cy6C^{Low}/Ly6G+. In some embodiments, PMN-MDSCs are identified as shown in FIG. 8G. In some embodiments, PMN-MDSCs are identified as shown in FIG. 8G with an additional step of selecting Ly6C^{Low}/Ly6G+ cells. In some embodiments, the cell is CD45+/CD11b+/Ly6C^{Low}/Ly6G+/Ly6E+. In some embodiments, the cell is identified by a surface expression profile comprising CD45+/CD11b+/Ly6C^{Low}/Ly6G+/Ly6E+. In some embodiments, Ly6C^{Low} is Ly6C−. In some embodiments, Ly6C^{Low} is Ly6C negative.

In some embodiments, a cell expression Ly6E is a cell highly expressing Ly6E. In some embodiments, a highly expressing cell is a Ly6E (hi) cell. In some embodiments, highly expressing is expressing above a predetermined threshold. In some embodiments, highly expressing is greater than 1 order of magnitude higher expression than a negative control. In some embodiments, highly expressing comprises expression higher than all negative cells. In some embodiments, the detection method is flow cytometry and highly expressing cells are cells that stain with a fluorochrome more highly than all negative cells. In some embodiments, negative cells are cells from a negative control. In some embodiments, a negative control is a secondary antibody control. In some embodiments, a negative control is cells that are known to be negative for Ly6E. In some embodiments, highly expressing is as defined in FIG. 3G. In some embodiments, highly expressing is as defined in FIG. 3H. In some embodiments, highly expressing is as defined in FIG. 8G. In some embodiments, highly expressing is expression that is at least twice the level of the lowest positively expressing cell. In some embodiments, highly expressing is expression that is at least 2, 3, 4, 5, 6, 7, 8, 9, or 10 times the expression of the lowest positively expressing cell. Each possibility represents a separate embodiment of the invention.

In some embodiments, the Ly6E expressing cell comprises an mRNA expression profile provided in Table 3. In some embodiments, the Ly6E expressing cell is characterized by an mRNA expression profile provide in Table 3. In some embodiments, the Ly6E cell is a neutrophil comprising or characterized by an expression profile provided in Table 3. In some embodiments, the Ly6E cell is an MDSC comprising or characterized by an expression profile provided in Table 3. In some embodiments, the Ly6E expressing cell is a neutrophil expressing Ly6E above a predetermined threshold.

In some embodiments, the Ly6E expressing cell is a human cell and comprises expression of at least one mRNA selected from Interferon induced protein with tetratricopeptide repeats 1 (IFIT1), ISG15 ubiquitin like modifier (ISG15), Interferon induced with helicase C domain 1 (IFIH1), HECT and RLD domain containing E3 ubiquitin protein ligase 5 (HERC5), Radical S-adenosyl methionine domain containing 2 (RSAD2), Interferon alpha inducible protein 6 (IFI6), Metallothionein 2A (MT2A), Epithelial stromal interaction 1 (EPSTI1), Cytidine/uridine monophosphate kinase 2 (CMPK2), Cap methyltransferase 1 (CMTR1), Interferon induced protein 44 like (IFI44L), DExH-box helicase 58 (DHX58), Serine rich and transmembrane domain containing 2 (SERTM2), and Interferon omega 1 (IFNW1). In some embodiments, expression is expression of at least 1, 2, 3, 4, 5, 6, 7, 8, 9, 10, 11, 12, 13 or 14 of IFIT1, ISG15, IFIH1, HERC5, RSAD2, IFI6, MT2A, EPSTI1, CMPK2, CMTR1, IFI44L, DHX58, SERTM2, and IFNW1. Each possibility represents a separate embodiment of the invention. In some embodiments, expression is expression of all of IFIT1, ISG15, IFIH1, HERC5, RSAD2, IFI6, MT2A, EPSTI1, CMPK2, CMTR1, IFI44L, DHX58, SERTM2, and IFNW1. In some embodiments, the human cell is a human MDSC.

In some embodiments, the Ly6E expressing cell is a mouse cell and comprises expression of at least one mRNA selected from Ifit1, C-×-C motif chemokine ligand 10 (Cxcl10), Guanylate binding protein 5 (Gbp5), Interferon gamma inducible protein 47 (Ifi47), Ifit2, Ifih1, Interferon gamma induced GTPase (Igtp), Schlafen 8 (Slfn8), Gbp3, Ubiquitin specific peptidase 18 (Usp18), Ring finger protein 213 (Rnf213), Proteasome 20S subunit beta 10 (Psmb10), Interferon induced protein 35 (Ifi35), Interleukin 18 binding protein (Il18 bp), Gbp7, Gbp9, Free fatty acid receptor 2 (Ffar2), Ifit3b, Triparite motif-containing 30C (Trim30c), Repulsive guidance molecule BMP co-receptor a (Rgma), Cmpk2, Olfactory receptor 56 (Olfr56), Microtubule interacting and trafficking domain containing 1 (Mitd1), Slfn9, Neurotrophin 5 (Ntf5), Tripartite motif containing 21 (Trim21), Interferon induced protein with tetratricpeptide repeats 1B like 1 (Ifit1bl1), Lymphocyte antigen 6 complex locus I (Ly6i), Poly(ADP-ribose) polymerase family member 12 (Parp12), and Ubiquitin conjugating enzyme E2 L6 (Ube2l6). In some embodiments, expression is expression of at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of Ifit1, Cxcl10, Gbp5, Ifi47, Ifit2, Ifih1, Igtp, Slfn8, Gbp3, Usp18, Rnf213. Psmb10, Ifi35, Il18 bp, Gbp7, Gbp9, Ffar2, Ifit3b, Trim30c, Rgma, Cmpk2, Olfr56, Mitd1, Slfn9, Ntf5, Trim21, Ifit1bl1, Ly6i, Parp12, and Ube2l6. In some embodiments, expression is expression of all of Ifit1, Cxcl10, Gbp5, Ifi47, Ifit2, Ifih1, Igtp, Slfn8, Gbp3, Usp18, Rnf213. Psmb10, Ifi35, Il18 bp, Gbp7, Gbp9, Ffar2, Ifit3b, Trim30c, Rgma, Cmpk2, Olfr56, Mitd1, Slfn9, Ntf5, Trim21, Ifit1bl1, Ly6i, Parp12, and Ube2l6. In some embodiments, the mouse cell is a mouse MDSC.

In some embodiments, the Ly6E expressing cell is a human cell and comprises increased expression of at least one mRNA selected from Sterile alpha motif domain containing 9 like (SAMD9L), MX dynamin like GTPase 1 (MX1), Signal transducer and activator of transcription 1 (STAT1), IFIT3, UBE2L6, IFIT5, PARP9, DExD/H-box helicase 58 (DDX58), Basic leucine zipper ATF-like transcription factor 2 (BATF2), PARP14, IFIT2, TRIM22, GBP5, Apolipoprotein L6 (APOL6), IFI16, REC8 meiotic recombination protein (REC8), (2′-5′-oligoadenylate synthetase like (OASL), TRIM5, Deltex E3 ubiquitin ligase 3L (DTX3L), Fc fragment of IgG receptor 1b (FCGR1B), STAT2, Fucosyltransferase 9 (FUT9), Serpin family G member 1 (SERPING1), GBP1, XIAP associated factor 1 (XAF1), Transmembrane protein 255B (TMEM255B), Zinc finger B-box domain containing (ZBBX), PARP12, ETS variant transcription factor 7 (ETV7) and LY6E. In some embodiments, the Ly6E expressing cell is a human cell and comprises increased expression of at least one mRNA selected from SAMD9L, MX1, STAT1, IFIT3, UBE2L6, IFIT5, PARP9, DDX58, BATF2, PARP14, IFIT2, TRIM22, GBP5, APOL6, IFI16, DDX58, REC8, OASL, TRIM5, DTX3L, FCGR1B, STAT2, FUT9, SERPING1, GBP1, XAF1, TMEM255B, ZBBX, PARP12, and ETV7. In some embodiments, the Ly6E expressing cell is a human cell and comprises increased expression of at least one mRNA selected from LY6E, IFIT3, IFIT1, IFIT2, STAT1, ISG15, STAT2, IFIT5, and IL1B. In some embodiments, the Ly6E expressing cell is a human cell and comprises increased expression of at least one mRNA selected from IFIT3, IFIT1, IFIT2, STAT1, ISG15, STAT2, IFIT5, and IL1B. In some embodiments, increased expression is as compared to the MDSC population in the sample. In some embodiments, increased expression is as compared to the neutrophil population in the sample In some embodiments, increased is as compared to a predetermined threshold. In some embodiments, increased is as compared to the average expression in the MDSC population in the sample. In some embodiments, increased is as compared to the average expression in the neutrophil population in the sample. In some embodiments, increased is as compared to LY6E negative cells. In some embodiments, increased is as compared to LY6E low cells. In some embodiments, increased expression is increased expression of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 of SAMD9L, MX1, STAT1, IFIT3, UBE2L6, IFIT5, PARP9, DDX58, BATF2, PARP14, IFIT2, TRIM22, GBP5, APOL6, IFI16, DDX58, REC8, OASL, TRIM5, DTX3L, FCGR1B, STAT2, FUT9, SERPING1, GBP1, XAF1, TMEM255B, ZBBX, PARP12, ETV7 and LY6E. Each possibility represents a separate embodiment of the invention. In some embodiments, increased expression is increased expression of all of SAMD9L, MX1, STAT1, IFIT3, UBE2L6, IFIT5, PARP9, DDX58, BATF2, PARP14, IFIT2, TRIM22, GBP5, APOL6, IFI16, DDX58, REC8, OASL, TRIM5, DTX3L, FCGR1B, STAT2, FUT9, SERPING1, GBP1, XAF1, TMEM255B, ZBBX, PARP12, ETV7 and LY6E. In some embodiments, increased expression is increased expression of at least 1, 2, 3, 4, 5, 6, 7, or 8 of IFIT3, IFIT1, IFIT2, STAT1, ISG15, STAT2, IFIT5, and IL1B. Each possibility represents a separate embodiment of the invention. In some embodiments, increased expression is increased expression of all of IFIT3, IFIT1, IFIT2, STAT1, ISG15, STAT2, IFIT5, and IL1B. In some embodiments, increased expression is increased expression of at least one of IFIT1, IFIT2, STAT1, and STAT2. In some embodiments, increased expression is increased expression of at least 1, 2, 3 or 4 of IFIT1, IFIT2, STAT1, and STAT2. Each possibility represents a separate embodiment of the invention. In some embodiments, increased expression is increased expression of all of IFIT1, IFIT2, STAT1, and STAT2.

In some embodiments, the Ly6E expressing cell is a mouse cell and comprises increased expression of at least one mRNA selected from Radical S-adenosyl methionine domain containing 2 (Rsad2), Isg15, Schlafen family member 5 (Slfn5), Gbp2b, Ly6e, Gbp2, Placenta associated 8 (Plac8), Parp14, Gbp7, Tumor necrosis factor (Tnf), Receptor transporter protein 4 (Rtp4), Proteasome 20S subunit beta 8 (Psmb8), Z-DNA binding protein 1 (Zbp1), Interferon simulated exonuclease gene 20 (Isg20), Ddx60, 2′-5′ oligoadenylate synthetase-like 2 (Oasl2), TRAF-type zinc finger domain containing 1 (Trafd1), Immunity-related GTPase family M member 1 (Irgm1), Chloride intracellular channel 4 (Clic4), Bone marrow stromal cell antigen 2 (Bst2), Transporter 1, ATP binding cassette subfamily B member (Tap1), Early growth response 3 (Egr3), Stat1, Stat2, Protease (prosome, macropain) activator subunit 2B (Psme2b), Signal peptide peptidase like 2A (Sppl2a), Ddx58, Interleukin 23 subunit alpha (Il23a), XIAP associated factor 1 (Xaf1), Deltex E3 ubiquitin ligase 3L (Dtx31), Parp10, Herc6, Torsin family 3 member A (Tor3a), Zinc finger containing ubiquitin peptidase 1 (Zufsp), N-myc and STAT interactor (Nmi), Trim30a, Trim56, NLR family CARD domain containing 5 (Nlrc5), Interferon regulatory factor 7 (Irf7), Parp9, 2′-5′-oligoadenylate synthetase 2 (Oas2), Immunity-related GTPase family M member 2 (Irgm2), Transporter 2, ATP binding cassette subfamily B member (Tap2)Tudor domain containing 7 (Tdrd7), Ubiquitin like modifier activating enzyme 7 (Uba7), Interleukin 15 receptor subunit alpha (Il15ra), T cell activation RhoGTPase activating protein (Tagap), Glypican 3 (Gpc3), Death domain associated protein (Daxx). In some embodiments, increased expression is increased expression of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 of Rsad2, Isg15, Slfn5, Gbp2b, Ly6e, Gbp2, Plac8, Parp14, Gbp7, Tnf, Rtp4, Psmb8, Zbp1, Isg20, Ddx60, Oasl2, Trafd1, Irgm1, Clic4, Bst2, Tap1, Egr3, Stat1, Stat2, Psme2b, Sppl2a, Ddx58, Il23a, Xaf1, Dtx31, Parp10, Herc6, Tor3a, Zufsp, Nmi, Trim30a, Trim56, Nlrc5, Irf7, Parp9, Oas2, Irgm2, Tap2, Tdrd7, Uba7, Il15ra, Tagap, Gpc3, Daxx. In some embodiments, increased expression is increased expression of all of Rsad2, Isg15, Slfn5, Gbp2b, Ly6e, Gbp2, Plac8, Parp14, Gbp7, Tnf, Rtp4, Psmb8, Zbp1, Isg20, Ddx60, Oasl2, Trafd1, Irgm1, Clic4, Bst2, Tap1, Egr3, Stat1, Stat2, Psme2b, Sppl2a, Ddx58, Il23a, Xaf1, Dtx31, Parp10, Herc6, Tor3a, Zufsp, Nmi, Trim30a, Trim56, Nlrc5, Irf7, Parp9, Oas2, Irgm2, Tap2, Tdrd7, Uba7, Il15ra, Tagap, Gpc3, Daxx.

In some embodiments, presence in the sample is presence above a predetermined threshold. In some embodiments, the predetermined threshold is at least 10, 15, 20, 25, 30, 35, 40, 45, 50, 55, 60, 65, 70, 75, or 80% of the neutrophils. Each possibility represents a separate embodiment of the invention. In some embodiments, the predetermined threshold is at least 10% of the neutrophils. In some embodiments, the predetermined threshold is at least 15% of the neutrophils. In some embodiments, the predetermined threshold is at least 20% of the neutrophils. In some embodiments, the predetermined threshold is at least 30% of the neutrophils. In some embodiments, the predetermined threshold is at least 40% of the neutrophils. In some embodiments, the predetermined threshold is at least 70% of the neutrophils. In some embodiments, the predetermined threshold is at least 80% of the neutrophils. In some embodiments, the predetermined threshold is at least 10, 15, 20, 25, 30, 35, 40, 45, 50, 55, 60, 65, 70, 75, or 80% of the MDSCs. Each possibility represents a separate embodiment of the invention. In some embodiments, the predetermined threshold is at least 10% of the MDSCs. In some embodiments, the predetermined threshold is at least 15% of the MDSCs. In some embodiments, the predetermined threshold is at least 20% of the MDSCs. In some embodiments, the predetermined threshold is at least 30% of the MDSCs. In some embodiments, the predetermined threshold is at least 40% of the MDSCs. In some embodiments, the predetermined threshold is at least 70% of the MDSCs. In some embodiments, the predetermined threshold is at least 80% of the MDSCs. In some embodiments, the neutrophils are MDSCs. In some embodiments, the neutrophils are a type of MDSC. In some embodiments, the MDSCs are PMN-MDSCs. It will be understood by a skilled artisan that the threshold can be in absolute terms, which are the number of the cells in the sample. However, as the sample size may vary the absolute number will also need to vary. Alternatively, the threshold can be measures as the percentage of all neutrophils, of all MDSCs or of all PMN-MDSCs that are Ly6E positive or highly expressing. In some embodiments, the method comprises determining the presence of MDSCs in the sample and determining the percentage of MDSCs that are Ly6E expressing, wherein a percentage above a predetermined threshold indicates the subject is suitable for the immunotherapy.

In some embodiments, presence of the Ly6E expressing cells indicates the subject is suitable for treatment. In some embodiments, absence of the Ly6E expressing cells indicates the subject is unsuitable for treatment. In some embodiments, presence of the Ly6E expressing cells indicates the subject is a responder. In some embodiments, a suitable subject is a responder. In some embodiments, an unsuitable subject is a non-responder. In some embodiments, absence of the Ly6E expressing cells indicates the subject is a non-responder. In some embodiments, the method further comprises treating a suitable subject with the immunotherapy. In some embodiments, the method further comprises treating an unsuitable subject with the immunotherapy and a pharmaceutical composition of the invention.

In some embodiments, a non-responder is a subject that is not responsive to the immunotherapy. In some embodiments, a non-responder is a subject with a non-favorable response to the immunotherapy. As used herein a “non-favorable response” of the cancer patient indicates “non-responsiveness” of the cancer patient to the treatment with the immunotherapy and thus the treatment of the non-responsive cancer patient with the immunotherapy will not lead to the desired clinical outcome, and potentially to a non-desired outcomes such as tumor expansion, recurrence and metastases. In some embodiments, the method further comprises discontinuing administration of the immunotherapy to a subject that is a non-responder.

In some embodiments, a responder is a subject that is responsive to the immunotherapy. In some embodiments, a responder is a subject with a favorable response to the immunotherapy. As used herein, a “favorable response” of the cancer patient indicates “responsiveness” of the cancer patient to the treatment with the immunotherapy, namely, the treatment of the responsive cancer patient with the immunotherapy will lead to the desired clinical outcome such as tumor regression, tumor shrinkage or tumor necrosis; an anti-tumor response by the immune system; preventing or delaying tumor recurrence, tumor growth or tumor metastasis. In this case, it is possible and advised to continue the treatment of the responsive cancer patient with the immunotherapy. In some embodiments, the method further comprises continuing to administer the immunotherapy to a subject that is a responder.

Pharmaceutical Compositions

By another aspect, there is provided a pharmaceutical composition comprising an Ly6E expressing cell.

In some embodiments, the pharmaceutical composition comprises a pharmaceutically acceptable carrier, excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition is formulated for administration to a human subject. In some embodiments, the composition is formulated for systemic administration. In some embodiments, the composition is formulated for intratumoral administration. In some embodiments, the composition is formulated for intravenous administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, intratumoral or intraperitoneal.

The dosage administered will be dependent upon the age, health, and weight of the recipient, kind of concurrent treatment, if any, frequency of treatment, and the nature of the effect desired.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, the pharmaceutical composition comprising a therapeutically effective amount of the cells. In some embodiments, a therapeutically effective amount is an amount sufficient to enhance response to the immunotherapy. In some embodiments, the therapeutically effective amount is a number of cells effective to treat the cancer in combination with the immunotherapy. In some embodiments, a therapeutically effective amount is an amount sufficient to convert a non-responder to a responder. In some embodiments, a therapeutically effective amount is an amount sufficient to increase the number of circulating Ly6E expressing cells above the predetermined threshold in a sample taken from blood. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the composition comprises a population of Ly6E expressing cells. In some embodiments, the cells are neutrophils. In some embodiments, the population expresses Ly6E above a predetermined threshold. In some embodiments, each cell of the population expresses Ly6E above a predetermined threshold. In some embodiments, the population is a subpopulation. In some embodiments, the subpopulation is within a population of immune cells. In some embodiments, the subpopulation is within a population of neutrophils. In some embodiments, the neutrophils expressing Ly6E above a predetermined threshold make up at least a predetermined threshold percentage of all neutrophils in the composition.

In some embodiments, the cells are naturally occurring neutrophils. In some embodiments, the cells are extracted neutrophils. In some embodiments, neutrophils are from a donor. In some embodiments, the neutrophils are induced neutrophils. In some embodiments, the neutrophils are generated neutrophils. In some embodiments, the neutrophils are GR1 positive neutrophils. In some embodiments, precursor cells are induced to produce the neutrophils for the composition. In some embodiments, cells of the composition are induced to increase expression of Ly6E. In some embodiments, composition comprises cells with induced Ly6E expression. In some embodiments, the induction comprises interferon stimulation. In some embodiments, the cells are interferon stimulated. In some embodiments, the interferon is interferon alpha. In some embodiments, the interferon is interferon gamma. In some embodiments, the interferon is a combination of interferon alpha and gamma. In some embodiments, the interferon is selected from interferon alpha, gamma and a combination thereof.

Methods of Treatment

By another aspect, there is provided a method of treating a subject suffering from a disease, the method comprising administering to the subject a pharmaceutical composition of the invention and an immunotherapy, thereby treating the subject.

By another aspect, there is provided a method of converting a non-responder to an immunotherapy to a responder, the method comprising administering to the non-responder a composition of the invention, thereby converting a non-responder to a responder.

In some embodiments, the disease is a disease treatable by the immunotherapy. In some embodiments, the disease is suitable to be treated by the immunotherapy. In some embodiments, the disease is caner. In some embodiments, the disease is a proliferative disease. In some embodiments, a disease is a disease such as is described hereinabove. In some embodiments, the subject is a non-responder to the immunotherapy. In some embodiments, the non-responder does not respond to the immunotherapy. In some embodiments, the non-responder is predicted not to respond to the immunotherapy. In some embodiments, the predicting is performance of a method of the invention. In some embodiments, the predicting comprises a method of the invention.

Kits

By another aspect, there is provided a kit comprising at least one reagent adapted to specifically determine an expression level of Ly6E.

In some embodiments, the Ly6E is human Ly6E. In some embodiments, the Ly6E is mouse Ly6E. In some embodiments, Ly6E is Ly6E protein. In some embodiments, the Ly6E comprises an amino acid sequence as provided hereinabove. In some embodiments, Ly6E is Ly6E mRNA. In some embodiments, the Ly6E nucleic acid sequence is provided hereinabove. In some embodiments, the nucleic acid is mRNA.

In some embodiments, the expression is selected from protein expression and mRNA expression. In some embodiments, the expression is protein expression. In some embodiments, the expression is mRNA expression. Reagents for detecting protein expression are well known in the art and include antibodies, protein binding arrays, protein binding proteins, and protein binding RNAs. Any reagent capable of binding specifically to Ly6E can be employed. As used herein, the terms “specific” and “specifically” refer to the ability to quantify the expression of one target to the exclusion of all other targets. Thus, for non-limiting example, an antibody that is specific to Ly6E will bind to Ly6E and no other targets. In some embodiments, the reagent is an antibody. In some embodiments, binding to a target and no other targets is binding measurably to a target and to no other targets. In some embodiments, binding to a target and no other targets is binding significantly to a target and no other targets. Reagents for detecting specific mRNAs are also well known in the art and include, for example, microarrays, primers, hybridization probes, and RNA-binding proteins. Any such reagent may be used. In some embodiments, the reagent is a primer. In some embodiments, the reagent is a pair of primers specific to Ly6E. It will be understood that a pair of primers that is specific will amplify the target and not significantly or detectably amplify other mRNAs. In some embodiments, the reagent is a nucleic acid molecule. In some embodiments, the reagent is an isolated oligonucleotide. In some embodiments, the isolated oligonucleotide specifically hybridizes to Ly6E or an mRNA of Ly6E. In some embodiments, the isolated oligonucleotide is no longer than 15, 20, 25, 30, 35, 40, 45 or 50 nucleotides in length. Each possibility represents a separate embodiment of the invention. In some embodiments, the isolated oligonucleotide hybridizes to only a portion of an mRNA of Ly6E. In some embodiments, the isolated oligonucleotide hybridizes to an mRNA of Ly6E with 100% complementarity. In some embodiments, the isolated oligonucleotide hybridizes to an mRNA of Ly6E with at least 90% complementarity. In some embodiments, the isolated oligonucleotide hybridizes to an mRNA of Ly6E with at least 95% complementarity. In some embodiments, the isolated oligonucleotide does not hybridize to an mRNA of a gene other than Ly6E with a complementarity of greater than 70, 75, 80, 85, 90, 95, 97, 99 or 100%. Each possibility represents a separate embodiment of the invention. In some embodiments, the isolated oligonucleotide does not hybridize to an mRNA of a gene other than Ly6E with 100% complementarity.

In some embodiments, the kit further comprises at least one reagent adapted to specifically determine the expression level of a control. In some embodiments, the control is a control such as described hereinabove. It will be understood that if the kit comprises reagents for determining protein expression of the factor, then the reagent for determining expression of the control would also determine protein expression. Similarly, for mRNA expression the reagents for the control would match the reagents for the factor. In some embodiments, the reagent for determining expression of the factor and the reagent for determining expression of the control are the same type of reagent.

In some embodiments, the kit further comprises detectable tags or labels. In some embodiments, the reagents are hybridized or attached to the labels. In some embodiments, the tag or label is a nucleic acid tag or label. In some embodiments, the nucleic acid tag or label is a primer. In some embodiments, the kit further comprises a secondary reagent for detection of the specific reagents. In some embodiments, the secondary reagents are non-specific and will detect all or a subset of the specific reagents. In some embodiments, the secondary reagents are secondary antibodies. In some embodiments, the secondary reagents are detectable. In some embodiments, the secondary reagents comprise a tag or label. In some embodiments, the tag or label is detectable. In some embodiments, a detectable molecule comprises a detectable moiety. Examples of detectable moieties include fluorescent moieties, dyes, bulky groups and radioactive moieties. In some embodiments, the kit further comprises a solution for rendering a protein susceptible to binding. In some embodiments, the kit further comprises a solution for rendering a nucleic acid susceptible to hybridization. In some embodiments, the nucleic acid is an mRNA. In some embodiments, the kit further comprises a solution for lysing cells. In some embodiments, the kit further comprises a solution for isolating plasma from blood. In some embodiments, the kit further comprises a solution for purification of proteins. In some embodiments, the kit further comprises a solution for purification of nucleic acids.

In some embodiments, a reagent is attached or linked to a solid support. In some embodiments, the reagent is non-natural. In some embodiments, the reagent is artificial. In some embodiments, the reagent is in a non-organic solution. In some embodiments, the reagent is ex vivo. In some embodiments, the reagent is in a vial. In some embodiments, the solid support is non-organic. In some embodiments, the solid support is artificial. In some embodiments, the solid support is an array. In some embodiments, the solid support is a chip. In some embodiments, the solid support is a bead.

In some embodiments, the kit comprises at least one reagent adapted to identify a neutrophil. In some embodiments, reagent binds a neutrophil specific protein. In some embodiments, the protein is a surface protein. In some embodiments, the protein is a neutrophil marker. In some embodiments, the reagent binds a protein not expressed by neutrophils. Neutrophil markers are well known in the art and any such markers can be used. Examples of markers can be found at biocompare.com/Editorial-Articles/577944-A-Guide-to-Neutrophil-Markers/among many other locations on the web. In some embodiments, the reagent binds specifically to CD45. In some embodiments, the reagent binds specifically to HLA-DR. In some embodiments, the reagent binds specifically to a lineage marker. In some embodiments, the reagent binds specifically to CD11b. In some embodiments, the reagent binds specifically to CD33. In some embodiments, the reagent binds specifically to CD14. In some embodiments, the reagent binds specifically to CD15. In some embodiments, the reagent binds specifically to CD66b. In some embodiments, at least one reagent is a panel of reagents adapted to identify a neutrophil. In some embodiments, the panel comprises at least 1, 2, 3, 4, 5, 6, 7, 8, 9 or 10 reagents. Each possibility represents a separate embodiment of the invention. In some embodiments, the panel of reagent comprises a reagent for specific identification of CD45, HLA-DR, CD11b, CD33, CD14, and CD15. In some embodiments, the panel of reagent comprises a reagent for specific identification of CD45, HLA-DR, CD11b, CD33, CD14, CD15 and at least one lineage specific marker. Lineage markers are well known in the art and specifically lineage markers related to immune cell development are also well known. A skilled artisan can thus select the lineage specific markers whose absence can identify a neutrophil. In some embodiments, the reagents are adapted for identification in a bodily fluid. In some embodiments, the bodily fluid is blood.

In some embodiments, the reagents for identifying neutrophils are antibodies. In some embodiments, the antibodies are fluorophore labeled antibodies. In some embodiments, each fluorophore of a different antibody is a distinct fluorophore. In some embodiments, distinct is distinctly identifiable. In some embodiments, identifiable is identifiable by flow cytometry. In some embodiments, the kit comprises uniquely identifiable antibodies against CD45, CD11b, CD33 and CD15. In some embodiments, the kit comprises uniquely identifiable antibodies against CD45, CD11b, CD33, CD14 and CD15. In some embodiments, the kit comprises uniquely identifiable antibodies against CD45, CD11b, CD33, HLA-DR and CD15. In some embodiments, the kit comprises uniquely identifiable antibodies against CD45, CD11b, CD33 and CD15 and antibodies against HLA-DR and CD14 wherein the antibodies against HLA-DR and CD14 are uniquely identifiable from the antibodies against CD45, CD11b, CD33 and CD15. In some embodiments, the antibodies against HLA-DR and CD14 are uniquely identifiable from each other. In some embodiments, the antibodies against HLA-DR and CD14 are not uniquely identifiable from each other. In some embodiments, the kit further comprises antibodies against at least one lineage specific marker. In some embodiments, the antibodies against the at least one lineage specific marker are uniquely identifiable from the antibodies against CD45, CD11b, CD33 and CD15. In some embodiments, the antibodies against the at least one lineage specific marker are uniquely identifiable from the antibodies against HLA-DR and CD14. In some embodiments, the antibodies against the at least one lineage specific marker are not uniquely identifiable from the antibodies against HLA-DR and CD14. It will be understood by a skilled artisan that since CD45, CD11b, CD33 and CD15 are positive selection markers they must be uniquely identifiable. However, as HLA-DR, CD14 and lineage markers should all be negative on neutrophils they can be uniquely identified, or all can be dumped in the same dump/undesired channel. Since any cell positive for these markers will be removed/excluded there is no need for specific unique identification. In some embodiments, uniquely identifiable is comprising a unique fluorophore. In some embodiments, a unique fluorophore has a unique emission spectrum. In some embodiments, a unique fluorophore has an emission spectrum that does not overlap or only minorly overlaps with the emission spectrum of another fluorophore in the kit. In some embodiments, a unique fluorophore has a unique excitation.

In some embodiments, the kit is for use in a method of the invention. In some embodiments, the kit is for use in determining suitability of a subject to be treated with an immunotherapy. In some embodiments, the kit further comprises instructions for use of the kit. In some embodiments, the instructions are for performance of a method of the invention.

In some embodiments, the instructions are for determining suitability of a subject to be treated with an immunotherapy.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Chemicals and Tumor Cell Cultures:

Anti-mouse PD-1 (clone RMP1-14, BioXCell) antibody for in vivo use was purchased from BioXCell. The antibody is given twice a week in a dose of 100 μg/mouse for up to 3-week period. As a control IgG antibody (BioXCell) was administered at the same dose.

4T1, EMT6 (murine breast carcinoma cell lines), CT26 (colon carcinoma), Renca (renal carcinoma) and LLC (murine Lewis lung carcinoma) were purchased from the American Type Culture Collection (ATCC, Manassas, Va., USA) and were used within 6 months of thawing. All the cell lines were grown in Dulbecco's modified Eagle's medium (DMEM, Sigma-Aldrich, Rehovot, Israel), and supplemented with 5% fetal calf serum (FCS), 1% L-glutamine, 1% sodium pyruvate, and 1% Pen-Strep-Neomycin in solution, (Biological Industries, Israel). All cells were cultured in humidified chamber in 5% CO₂ at 37° C. Cells were routinely tested to be mycoplasma-free.

The generation of responsive and non-responsive tumors to immunotherapy. Tumor cell lines were generated to respond to immunotherapy or to be resistant, based on the resistance characteristics of the parental cell. Previous studies demonstrated that immunotherapy is active in tumors with higher mutational load, therefore contributing to tumor immunogenicity. In order to generate responsive cell lines, cells underwent mutagenesis using 1-methyl-3-nitro-1-nitrosoguanidine (MNNG). Selected clones have been validated in vivo for their response to ICI therapy. Specifically, responsive clones for 4T1 parental (4T1^{P}) were successfully generated, namely its mutagenized counterpart cell 4T1 MNNG (4T1^{M}). Similarly, CT26, Renca, LLC and EMT6 parental and mutagenized cells were obtained. Of note, several cell lines, e.g., EMT6 demonstrates spontaneous response to ICI in a percentage reaching almost 30%. They have been used in this experimental setting without mutagenesis.

Murine tumor models. The use of animals and experimental protocols were approved by the Animal Care and Use Committee of the Technion. Female BALB/c and C57Bl/6 mice (8 weeks of age) were purchased from Envigo, Isra-el. Mixed background mice were created by backcrossing female C57Bl/6 and CBA female mice with pure C57Bl/6 male mice for 5 generations. All mice were maintained under specific pathogen-free conditions in the animal facility. 4T1P, 4T1M and EMT6 (5×105/50 μL in serum free medium) were ortho-topically injected into the mammary fat pad of 8-10-week-old female BALB/c mice. RencaP, RencaM, LLCP and LLCM (5×105/50 μL in serum free medium) were subcutaneously injected into the flanks of 8-10-week-old female BALB/c and C57Bl/6 mice, respectively. Mice were randomly grouped before therapy. In all experiments, when tumors reached ˜50 mm3 mice were treated with anti-mouse anti-PD-1 (clone RMP1-14, BioXCell) antibody. The antibody was given twice a week in a dose of 100 μg/mouse for up to 2-week period. The control groups were injected with IgG antibody (BioXCell). Tumor volume was measured twice a week with Vernier caliper and calculated by using the formula width 2×length×0.5. When tumor size reached endpoint (approximately 1,000 mm3) the experiment was terminated and mice were sacrificed, unless indicated otherwise.

The Establishment of Multi-Model Approach to Search for Predictive Biomarkers for Immunotherapy.

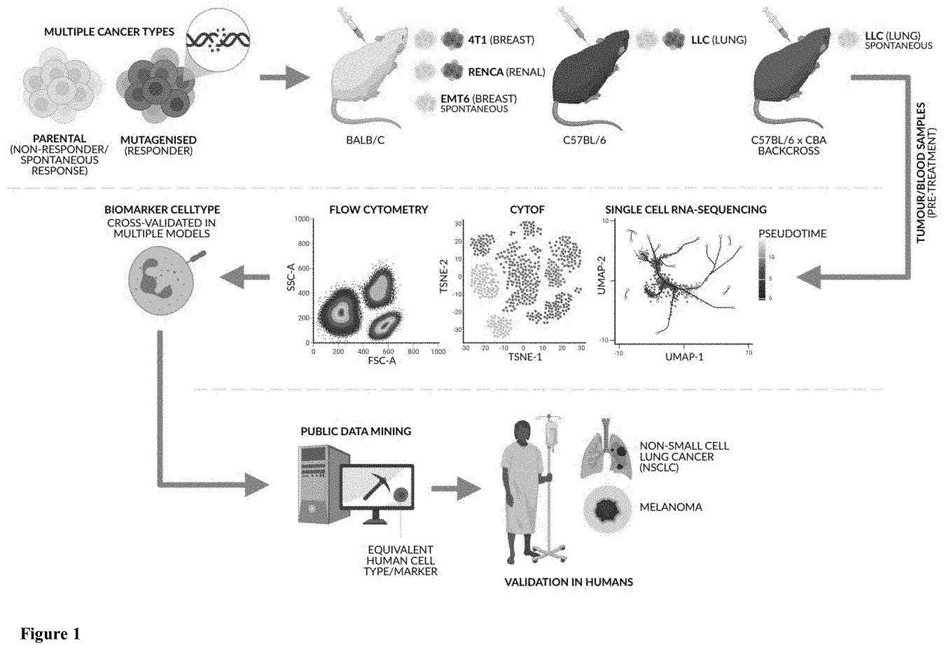

One of the major obstacles in immune-oncology is the use of mouse models to study immunotherapy. Herein is used a multi-model approach to search for biomarkers for ICI therapy (FIG. 1). In this approach multiple cancer types (breast, lung, renal cancers) are used, three different mouse strains (BALB/c, C57Bl/6 and C57Bl/6×CBA backcrossed) are used, and multiple clones of the same tumor cell lines (4T1 murine breast carcinoma, LLC lung carcinoma and Renca renal cell carcinoma, all parental clones were obtained from the ATCC) are used (Table 1).

TABLE 1
List of tumor models used

| | | | | Model |
|---|---|---|---|---|
| Cell Line | Clones | Cancer Type | Mouse Strain | Category |
| 4T1 | 4T1_{P}, | Breast Carcinoma | BALB/c | Mutagenesis |
| | 4T1_{M} | | | |
| RENCA | RENCA_{P}, | Renal | BALB/c | Mutagenesis |
| | RENCA_{M} | Adenocarcinoma | | |
| LLC | LLC_{P}, | Lung Carcinoma | C57BL/6 | Mutagenesis |
| | LLC_{M} | | | |
| EMT6 | EMT6_{P} | Breast Carcinoma | BALB/c | Spontaneous |
| LLC | LLC_{P} | Lung Carcinoma | C57BL/6 | Backcross |
| | | | x CBA | |

The initial three rows (mutagenesis) are tumor-dependent models. The remaining rows are host-dependent models. P=parental cells, M=mutagenized cells.

Mutagenized model: Cell line pairs were generated comprising a clone that is responsive to anti-PD1 generated from a non-responsive parental cell line. The responsive clones were generated through mutagenesis (see below), therefore mimicking mutational load—a clinically relevant metric for immuno-therapy response. This process provides pairs of cells originating from the same cell line, allowing a biologically relevant comparison.

Spontaneous model: A tumor cell line that displays a natural, spontaneous response to anti-PD1 (EMT6 cell line) was used. This model mimics a host dependent mechanism of response to immunotherapy.

Backcrossed model: A mixed background strain (outbreed) was generated. Specifically, C57Bl mice were bred with CBA mice to create an F1 generation. F1 progeny are unable to grow syngeneic C57Bl/6 tumors. These mice were backcrossed with inbred C557Bl/6 mice for 5 generations, as opposed to the standard 10. The resulting mice are compatible with C57Bl/6 syngeneic cell lines but retain enough heterogeneity to drive a variable host-dependent response to anti-PD1.

Using this multi-model approach, tumors or blood can be harvested at baseline (the pre-treatment stage) and subjected to high resolution single cell assays (e.g., single cell RNA sequencing [scRNA-seq] or mass cytometry [CyTOF]) to identify cell states that differentiate between eventual responders and non-responders (FIG. 1). Herein, to demonstrate the use of this approach, scRNA-seq was performed on 4T1 tumor bearing mice and a specific cell type that was enriched in responders was identified. Subsequently, this potential cellular biomarker was validated in all other models—establishing its ability to predict immunotherapy response in mice regardless of the underlying mechanism.

To translate the use of this cellular biomarker into humans, a functionally equivalent cell state can be identified through public data mining (FIG. 1). Functional equivalence is superior to the use of direct orthologues (e.g., Gene-A in both mouse and human) as they may not necessarily mark the same cell state in a different species. Here, published scRNA-seq data was analyzed from the blood of non-small cell lung carcinoma (NSCLC) patients to identify cells undergoing similar biological processes to the cells identified in mice.

Subsequently, the cellular biomarker was validated in a separate retrospective cohort of NSCLC patients treated with ICI-based therapy.

Single cell suspension preparation. Tumors were removed from mice, cut into small pieces and transferred to genteleMACS™ C tubes (Miltenyi Biotec, Germany) containing 5 ml of RPMI medium supplemented with 20% FCS, 1% L-glutamine, 1% sodium pyruvate, and 1% Pen-Strep-Neomycin. Tumors were subjected to homogenization using genteleMACS™ dissociator (Miltenyi Biotec, Germany), supplemented with 32 mg/ml dispase II (Godo Shusei Co., Ltd, Tokyo, Japan) and 38 mg/ml collagenase type 1 (Worthington Biochemical Corp, Lakewood, N.J., USA) and were incubated for 1 hour at 37° C. in a shaker incubator. Tumor homogenates were applied on cell strainers (70 μl mesh size) placed on a 50 ml tube and subsequently were centrifuged at 470×g for 5 min. Pellets containing the isolated single cells were resuspended with PBS to the required volume for further experimental procedures and analysis.

Tumor lysate preparation and protein measurement. 4T1P and 4T1M tumor tissues were placed in a 1.6 mL tube containing RIPA buffer (5M NaCl, 0.5M EDTA pH=8, 1M Tris pH=8, 1% NP-40, 10% sodium deoxycholate, 10% SDS) and protease inhibitor cocktail (1:100, Sigma-Aldrich, St Louis, Mo., USA). Stainless steel beads (SSB14B, Next Advance, New York, USA) were added and tumor tissue was homogenized using the Bullet Blender Tissue Homogenizer (Next Advance) according to the manufacturer's protocol. The homogenate was centrifuged, and supernatant was collected. The protein concentration of the tumor lysates was determined using Pro-tein Assay Dye Reagent Concentrate (Bio-Rad, California, USA). The quantification of INFg and TNFa was carried out by using LEGENDplex Mouse Th1/Th2 Panel (BioLegend, San Diego, Calif., USA), in accordance with the manufacturer's instructions. In addition, IFNa was quantified by specific ELISA (&D Systems, Minneapolis, Minn., USA) according to the manufacturers' instructions.

Single cell RNA sequencing on Gr1+ cells. The evaluation of neutrophil/MDSC subpopulations in responsive and non-responsive tumors was performed by single-cell RNA sequencing (scRNA-seq). Briefly, 4T1p and 4T1m tumors were prepared as single cell suspensions. Subsequently, GR1+ cells were isolated by positive isolation (EasySep Mouse PE, Biolegend) from responsive and non-responsive 4T1 tumors to anti-PD1 therapy. The cells were than washed in PBS with 0.04% BSA and resuspended in 1000 cells/μL PBS. RNA was extracted and immediately was acquired by the 10× Genomics single cell sequencing system. Bioinformatic analysis was further carried out to profile the heterogeneous cell population of neutrophil/MDSCs with massive throughput digital gene expression on a cell-by-cell basis. Changes in specific subpopulation of cells were plotted and validated by flow cytometry based on unique expressed surface markers, as outlined hereinbelow.

Flow cytometry acquisition and analysis. Validation of cell subpopulation in tumors and peripheral blood was carried out as follows. Cells from tumors after the tumor underwent single cell suspension or peripheral blood after the samples underwent red blood cell lysis, were immunostained for the following surface markers: Murine and human granulocytic population were defined as CD45+/CD11b+/Ly6CLowLy6G+ and CD45+/Lin-HLA-DR−/CD33+CD11b+/CD14-CD15+, respectively, as previously described (Ref). In addition, immune cells were defined based on the following surface markers: NK cells (CD45+/NKp46+), B cells, (CD45+/B220+), cytotoxic T cells, (CD45+/CD3+/CD8+), T helper cells (CD45+/CD3+/CD4+), and monocytes (CD45+/CD11b+/Ly6C+/Ly6Glo). All monoclonal antibodies were purchased from BD Biosciences, R&D systems, and Macs Militenyi Biotec. Ly6E antibodies from mouse and human were purchased from Novusbio, Novus Biologicals, CO, USA, and Creative Biolabs, NY, USA, respectively. All antibodies were used in accordance with the manufacturers' instructions. At least 300,000 events were acquired using a Fortessa flow cytometer and analyzed with FlowJo V.10 software (FlowJo, Ashland, Oreg., USA).

Adoptive transfer of Ly6E(hi) neutrophils. GR1 cells were isolated (positive isolation, EasySep Mouse PE, Biolegend) from the spleens of 4T1 tumor bearing mice and cultured overnight with 5% medium containing INFa and IFNg (10 ng/ml, Bio-Legend, San Diego, Calif., USA). Subsequently, cells were collected, centrifuged and washed twice with PBS. Ly6E+ neutrophils were analyzed by flow cytometry and by RT-qPCR as described below. For the adoptive transfer procedure, the Ly6E+ neutrophils obtained as described above, were intravenously injected to recipient 50 mm3 4T1 tumor bearing mice, 4 hours before each time the mice were injected with anti-PD1 or IgG control. Tumor volume was measured twice a week. When tumor reached endpoint the mice experiment was terminated.

Real-Time quantitative PCR (RT-qPCR). RNA was extracted from the in vitro Ly6E+ induced cells using Total RNA Purification Kit (Norgen, Ontario, Canada). cDNA was synthesized using High-Capacity cDNA Reverse Transcription Kit (Applied Bio-systems, California, USA). RT-PCR reaction was performed using SYBR Green Master Mix and run in CFX Connect Real-Time PCR Detection System (Bio-Rad, California, USA). Analysis was performed using ΔΔCt method. Primers are listed in Table 2.

TABLE 2
List of primers used for RT-qPCR.

| Gene | Forward (SEQ ID NO:) | Reverse |
|---|---|---|
| mTNFα | CTGAACTTCGGGGTGATCGG (1) | GGCTTGTCACTCGAATTTTG |
| | | AGA (2) |
| mCXCL1 | CTGGGATTCACCTCAAGAACAT | CAGGGTCAAGGCAAGCCTC |
| | C (3) | (4) |
| mIL1α | TCTCAGATTCACAACTGTTCGT | AGAAAATGAGGTCGGTCTCA |
| | G (5) | CTA (6) |
| mIL23α | CAGCAGCTCTCTCGGAATCTC | TGGATACGGGGCACATTATT |
| | (7) | TTT (8) |
| mSaa3 | TGCCATCATTCTTTGCATCTTG | CCGTGAACTTCTGAACAGCC |
| | A (9) | T (10) |
| mCCL3 | TGTACCATGACACTCTGCAAC | CAACGATGAATTGGCGTGGA |
| | (11) | A (12) |
| mCCL6 | AAGAAGATCGTCGCTATAACC | GCTTAGGCACCTCTGAACTC |
| | CT (13) | TC (14) |

Single cell RNA-seq alignment and pre-processing. Raw, Illumina base calls (BCLs) were demultiplexed and the resulting FASTQ files were aligned to the mm10 (GRCm38, Ensembl 93) murine reference genome and normalized for sequencing depth using CellRanger [v 5.0.1] to generate expression matrices. 82.8-85.7% of reads mapped to the transcriptome across all samples. A median of 3,252 and 2801 unique molecular identifiers (UMI) per cell for NR IgG and R_IgG were observed respectively. R [v4.1.0] and Python [v3.8.5] were used for all downstream analyses. Genes expressed in <10 cells were discarded. High-quality cells were retained by excluding: (i) cells expressing <500 or >5000 unique genes and (ii) cells with a mitochondrial UMI4 proportion of >10%—yielding 4711 cells and a total of 14214 detectable genes. SCTransform [v0.3.2], accessed via Seurat [v4.0.3], was utilized to normalize and scale the data, select 3000 variable features and linearly regress out any remaining influence of mitochondrial UMI % on downstream analyses. SCTransform specifically mitigates technical factors, but retains biological heterogeneity, improving downstream analysis.

Classification of cell types. To classify all 4711 cells in an unsupervised manner, SingleR [v1.6.1] was utilized to compare the transcriptome of each cell to a dual-reference of sorted microarray (ImmGen) and mouse RNA-seq data provided by celldex [v1.2.0]. 34 cells (1.180%) with ambiguous or poor-quality classifications were discarded—as determined by the SingleR prunescores function set to a threshold of 3 absolute mean deviations. Contaminating cells (i.e., non-GR1+, or non-myeloid cells) were discarded and classifications were broadly verified in a supervised manner using known myeloid (Cd11b, Cd11c), monocytic (Ly6c, Cs1fr, MHCII) and granulocytic (Ly6g, Cs3fr, Csf1) marker genes (1811 monocytic, 2866 granulocytic cells in total).

Dimensionality reduction, unsupervised clustering and differential abundance analysis. Data from all samples was aggregated and, as calculated by the Seurat [v4.0.3] functions RunPCA and RunUMAP respectively (default parameters), the top 3000 variable features and 25 principal components were utilized to generate a uniform manifold approximation and projection (UMAP) for visualization of the data. To assess globular, cellular heterogeneity, transcriptionally distinct cell states were defined by shared k-nearest-neighbor (s-KNN) analysis and Louvain-Jaccard clustering via the Seurat [v4.0.3] functions FindNeighbors and FindClusters respectively, using a resolution of 0.75. Cellular neighborhoods displaying differential abundance between conditions were defined by DASeq [Ref] [v1.0.0] using the top 10 principal components and k-values of [50-1000] at 50 stepwise intervals. Non-significant neighborhoods were discarded, as determined by a random permutations test.

Data visualization. Gene expression and UMAPs were visualized using dynplot [v1.1.1] or as binned, hexplots generated by schex [v1.6.3]. Where noted, MAGIC [v2.0.3] was used to impute the data, based on an automatically calculated level of diffusion (parameter t=auto). Imputed data was solely used for the purposes of visualization.

Differential gene expression analysis. All differentially expressed genes were identified using the scRNA-seq-specific tool MAST [v1.18.0] accessed via the Seurat [v4.0.3] FindMarkers function. Significance was assessed by calculating adjusted FDR p-values using the Bonferroni correction method and a gene was considered to be differentially expressed if its log 2 fold-change was >±0.35.

RNA-velocity and trajectory inference. Using velocyto [v0.17], the fractions of unspliced:spliced mRNAs were computed for all ˜20,000 genes in the raw FASTQ data. The resulting LOOM files were imported to Seurat [v4.0.3] and pre-processed as above. RNA-velocity vectors were dynamically modeled using scvelo [v0.2.4] under default parameters (number of principal components=30, number of neighbors=30). To map the differentiation hierarchy of granulocytes, partition-based graph abstraction (PAGA, via scanpy [v1.8.0] [Refs]) was combined with velocity-inferred directionality to infer trajectories using the scvelo function scvelo.tl.paga. Optimal topology was ensured by discarding all non-significant cluster-to-cluster connections (connectivity score <0.1) and the resulting trajectories were projected back onto the original UMAP using dynplot [v1.1.1].

Gene modules and pathway analysis. To identify genes with pseudotime-associated patterns of expression, negative binomial generalized additive models (NB-GAMs) were fit to ˜14,000 genes and the significance of association was statistically tested by tradeSeq [v1.6.0]. NB-GAMs were fit using the parameter nknots=6—a conservative estimate, as determined by the tradeSeq function, evaluateK, to avoid overfitting. Expression patterns were binned (n=20) along pseudotime and clustered via clusterExperiment [v2.12.0] to de-fine distinct gene modules. To characterize each module, over-representation tests were performed using clusterProfiler [v4.0.0] and gene-lists from the HALLMARK database (biological processes) and msigdbr [v7.4.1] (category=C3, transcription factors). The latter determines which, if any, transcription factors (Tfs) regulate the genes present in each module. Only significantly enriched (FDR <0.01, Bonferroni correction method) processes and TFs were retained.

Trajectory alignment. To compare trajectory lineages, a common pseudotemporal axis was defined using cellAlign [v0.1.0]—set to default, globalAlignment parameters as specified here: github.com/shenorrLab/cellAlign. In brief, inferred pseudotime values (defined by PAGA/RNA velocity), and the normalized expression values of all genes in modules common to both lineages were utilized to align the trajectories across 200 interpolated points and module enrichment values were averaged at corresponding, aligned pseudotime values.

Human analysis. Raw, scRNA-seq expression matrices were downloaded from the GEO Omnibus database (GSE127465) (N=8, blood, NSCLC cancer patients at baseline). Data was imported into Seurat [v4.0.3] and pre-processed using SCTransform [v0.3.2] with identical filtering criteria to mouse—yielding 13403 cells and a total of 22413 detectable genes. To classify all 13404 cells in an unsupervised manner, SingleR [v1.6.1] was utilized to compare the transcriptome of each cell to the Human Primary Cell Atlas reference, as provided by celldex [v1.2.0]. 1701 (14.4%) non-immune cells or cells with ambiguous or poor-quality classifications were excluded. Human-specific gene-lists from the HALLMARK database, as accessed in R via msigdbr [v7.4.1], for (i) interferon_alpha_response (ii) interferon_gamma_response and (iii) tnfa_signalling_via_nfkb were combined to generate a functional signature representative of Ly6E(hi) neutrophils. The enrichment of each, individual cell for the resulting signature was scored using the Seurat [v4.0.3] ssGSEA-like function, AddModuleScore.

Blood collection from cancer patients. The human study was approved by the ethic committee at Sheba medical center, Tel Hashomer, Israel, and by Rambam Medical center, Haifa, Israel, after patients signed an informed consent. Blood was drawn at baseline, before immunotherapy, from non-small cell lung cancer patients (n=34), and for melanoma patients (n=16). Patients' characteristics are indicated in Tables 4 and 5, respectively. Peripheral blood mononuclear cells (PBMCs) were isolated from ficoll tubes and stored in freezing medium (Nutrifreez D10, Biological industries, Israel) at −80° C., until further analyzed. PBMCs were then thawed and analyzed by flow cytometry using a mixture of antibodies indicated above. Response rate was determined by RECIST at 3 and/or 6 months and was evaluated in all patients and correlated with the levels of Ly6E+ neutrophils cells.

Statistical analysis. All statistical tests were performed in R [v4.1.0]. Statistical, pairwise comparisons for ELISA, LEGEND-plex and Flow Cytometry data were performed using unpaired, two-sample Mann-Whitney tests (R function: wilcox.test). Two-sample Kolmogorov-smirnov tests (R function: ks.test) were utilized to compare tumor growth curves. Mice were randomized before tumor implantation. The analysis of the results was performed blindly. At least 5 mice per group were used in order to reach statistical power considering a Gaussian distribution. Where appropriate (e.g., differential gene expression analysis), p-values were adjusted using the Bonferroni correction method to control for type I error rates i.e. false discovery rate (FDR). In all cases, significant differences were considered if p-values or FDR were <0.01. The number of samples or independent experiments are indicated in the text. For NSCLC and melanoma patients, the investigators were blinded to allocation (i.e., RECIST categories) during experiments and outcome assessment. Co-variates including age, sex and stage were not controlled for.

### Results

### Example 1: A Combination of Strains and Tumors Generate Diverse Responses to Immunotherapy

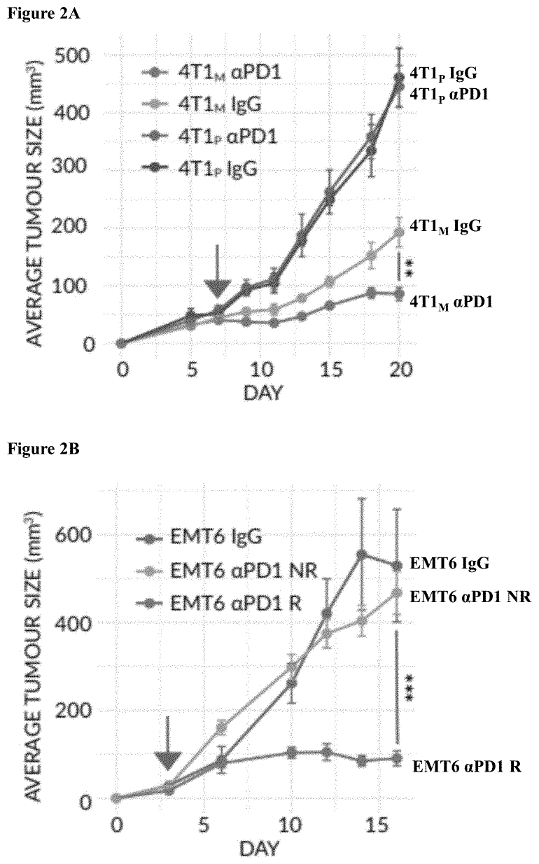

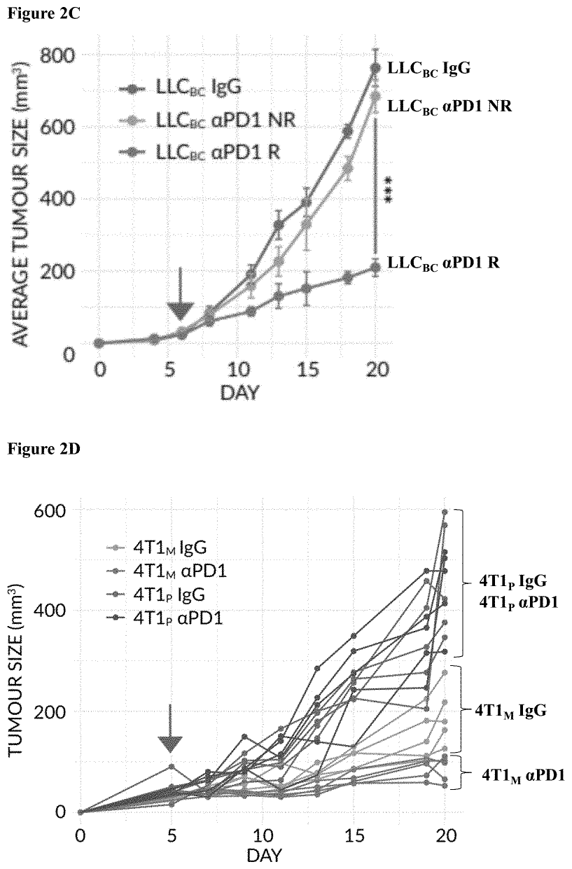

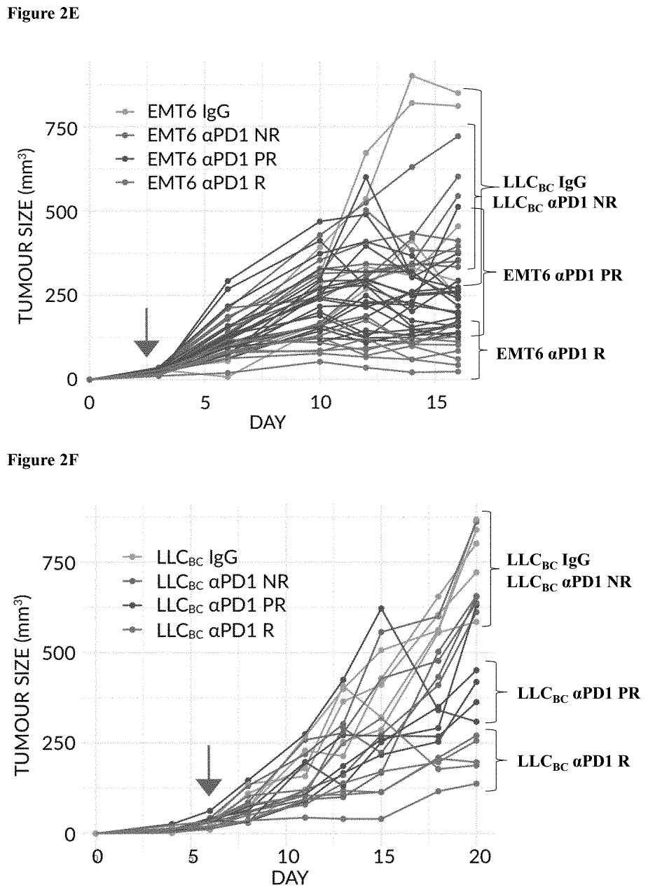

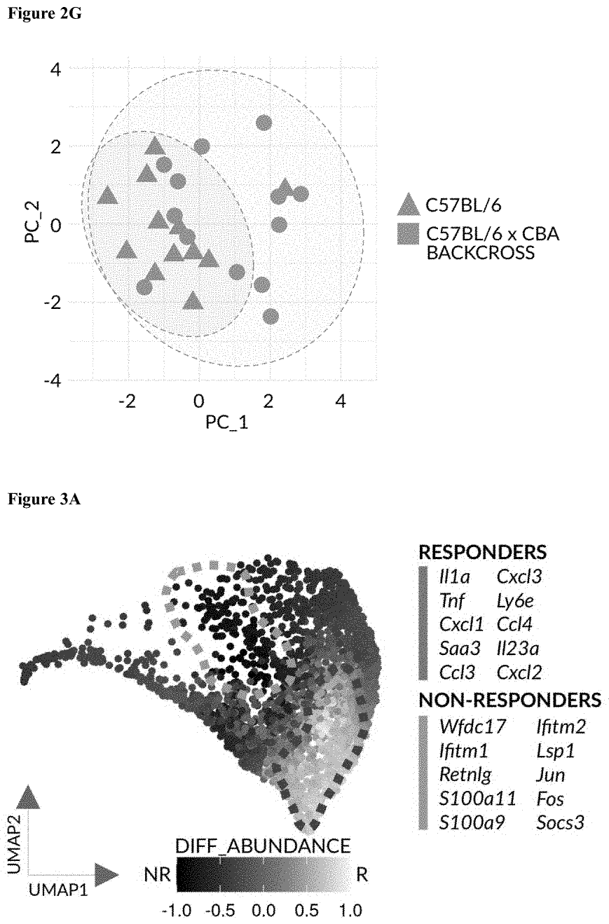

Translating preclinical biomarkers into clinical practice faces significant obstacles, in part due to the lack of appropriate models. For example, recent studies often employ a single mouse strain implanted with a single tumor type using cell lines. Such an approach will likely fail to recapitulate the complex mechanisms of immunotherapy response, including host effects and mutational load. Therefore, both sources of variability were modeled using a multi-model approach including diverse tumor types and mouse strains in which one can distinguish response and non-response to immunotherapy (FIG. 1). Specifically, mutational burden was introduced via cell line mutagenesis (FIGS. 2A and 2D), and host effects were measured in two different ways: a cancer cell line displaying a spectrum of spontaneous responses to ICI (FIGS. 2B and 2E); and a mixed mouse strain which displays variability in immune cell composition (FIGS. 2C and 2F-G). By cross-validating any candidate biomarker across these diverse models, one is able to discover a biomarker for a wider patient population.

### Example 2: The Validation of Responsive and Non-Responsive Tumors to Anti-PD1 Therapy

A prerequisite of biomarker discovery at baseline (i.e., pre-treatment) is the use of a stable and predictable model whose response outcomes are known apriori. Therefore, initial efforts were focused on a 4T1 mutagenized model whose response displays consistency (FIGS. 2A and 2D). 4T1 murine breast carcinoma cells that are considered to be resistant to ICI therapy, underwent mutagenesis by exposure to 1-methyl-3-nitro-1-nitrosoguanidine (MNNG) which induces DNA breaks and contributes to higher mutational loads. Selected clones were validated in vivo where 4T1 parental (4T1P) cells and 4T1-MNNG (4T1M) cells were implanted into the mammary fat pad of BALB/c mice. When tumors reached 50 mm³, treatment with anti-PD-1 was initiated, and tumor growth was monitored over time (FIG. 2A). As can be seen in FIG. 2A, 4T1^{M} tumors developed slower than 4T1^{P} tumors, implying the involvement of the immune system against tumor cells. Most importantly, 4T1^{M} cells displayed a partial response to anti-PD-1 therapy in contrast to 4T1^{P} tumors which were resistant.

### Example 3: Altered Ly6E(Hi) Neutrophil Frequency in Responsive and Non-Responsive Tumors

Enrichment of specific neutrophil clusters as a function of response was observed (FIG. 3A). These clusters were associated with abundant, enriched expression of 192 different genes (>1.5 fold change), including 30 genes with a >2 fold-change—providing a large pool of candidates. However, it was reasoned that a successful biomarker must fit the following criteria: (a) a cell surface marker to be analyzed by simple flow cytometry, (b) a mechanistic understanding, and ideally (c) its expression is found in a metastable or highly differentiated cell state as opposed to a transient state that may be difficult to consistently detect. Thus, in order to select a candidate gene, trajectory analysis was performed, and the RNA velocity of the cells was calculated. A branched trajectory was observed that converges upon a single cell state (FIG. 3B). Consistent with the inferred direction of this trajectory, expression of known progenitor genes was observed at earlier pseudo time values, suggesting the trajectory is biologically relevant. Since both branches of the trajectory are differentiating toward the same cell state, it was hypothesized that they may share a common differentiation program. The trajectory branches were therefore aligned, and modules of genes shared between both branches that change with pseudo time were defined. By calculating the density of cells across the aligned trajectory, and characterizing each module, it was observed that non-responders typically fail to progress beyond an apoptotic and inflammatory state, while responders differentiate further to a state marked by response to IFN and NFkB/TNFa signaling, suggesting exposure to IFN is a major driving force behind this differential progression (FIG. 3C). When tested in vitro at the protein level, a similar correlation was observed, validating the scRNAseq results (FIG. 3D). Thus, Ly6E (FIG. 2E), a known IFN-stimulated genes with a high pseudo-time value, was confirmed as a prime biomarker candidate for responding tumors. Overall, a high frequency of Ly6E(hi) neutrophilic cells in the tumor correlates with immunotherapy response, as validated by flow cytometry (FIG. 3F). An example of the cutoff drawn for considering neutrophil as having “high” Ly6E is provided in FIG. 3G, and this cutoff is used to compare Ly6E(hi) neutrophils in mice bearing tumors. (FIG. 3H).

### Example 4: Validation of Ly6E(Hi) Neutrophils in Tumor and Peripheral Blood of Mice Bearing Responding Tumors

To validate the levels of cells at the protein level, 4T1^{P} and 4T1^{M} cells (non-responsive and responsive to anti-PD-1, respectively) were implanted into BALB/c mice. Tumor growth was assessed regularly, and as expected tumor growth of responsive tumors to anti-PD-1 was slowed as compared to non-responsive tumors (FIG. 2A).

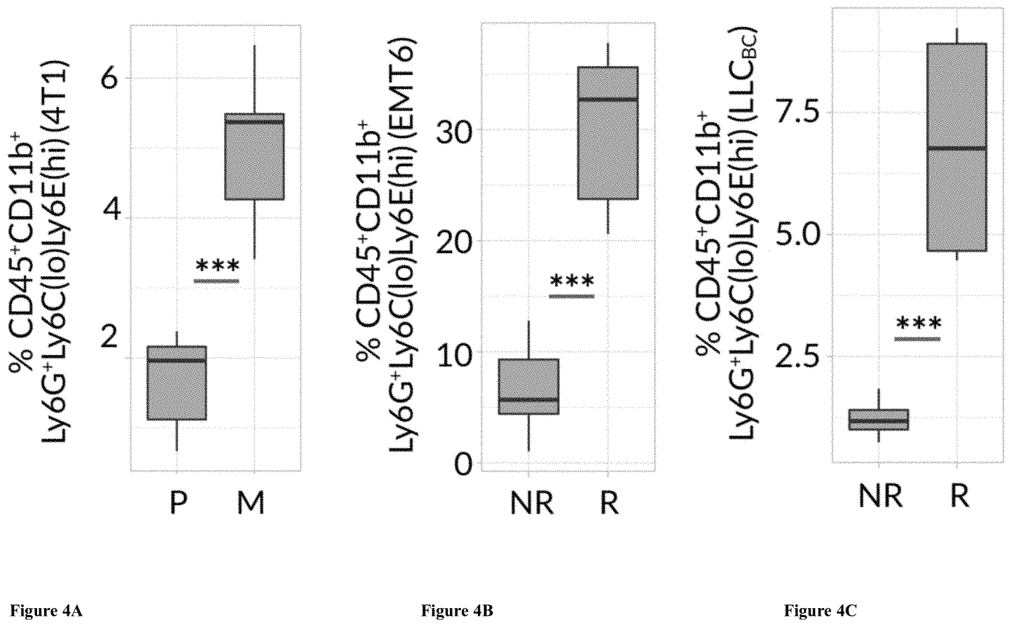

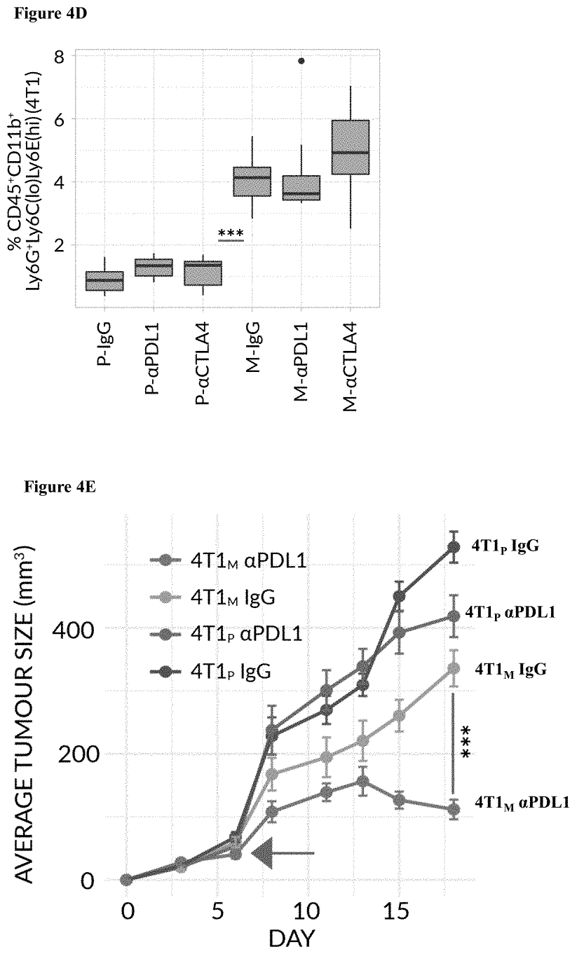

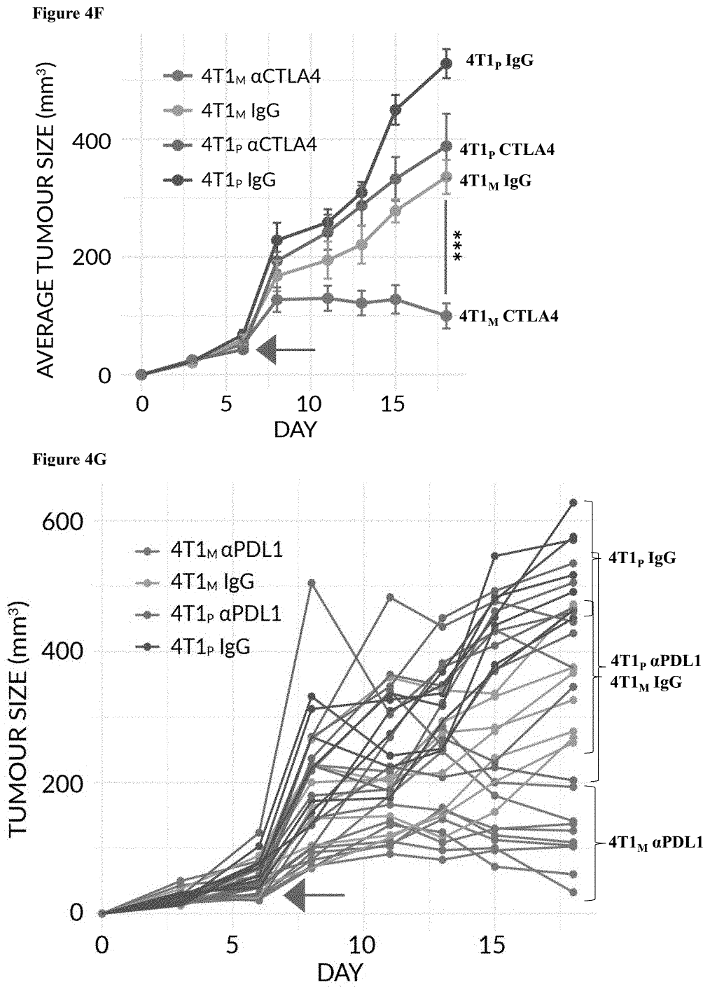

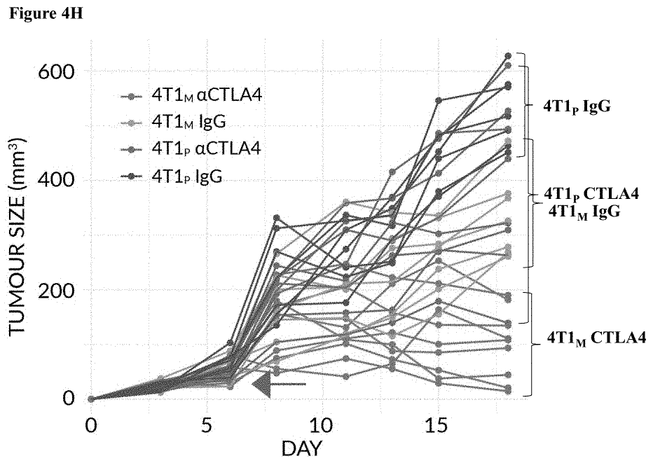

The gold standard of clinical utility is a liquid biopsy. To evaluate the potential of LY6E(hi) neutrophils as a blood-borne biomarker, their frequencies were analyzed in blood taken from multiple models (mouse strains) and tumor types (breast, lung, and renal cancers) (FIG. 1), at baseline. Further, at the experimental end point, tumors were removed and prepared as single cell suspension and blood was drawn by cardiac puncture. Flow cytometry analysis using the marker composition of CD45+/CD11b/Ly6C^{Low}/Ly6G+/Ly6E+ cells was carried out (FIG. 4A-C). Ly6E(hi) neutrophils were found to be highly enriched within the blood of mice bearing tumors responsive to anti-PD1 (FIG. 4A-C), demonstrating that these cells are a universal marker for anti-PD1 therapy regardless of whether or not the response is tumor- or host-dependent. These results taken together demonstrate that the level of Ly6E(hi)neutrophils in peripheral blood is predictive of responsiveness to PD-1 therapy.

These results were recapitulated with two other ICIs: anti-PD-L1 and anti-CTLA4. 4T1^{P} and 4T1^{M} cells (non-responsive and responsive to anti-PD-1, respectively) were implanted into the mammary fat pad of BALB/c mice. For the sake of completeness, when tumors reached an average size of 50 mm\hat{ }3 blood was drawn and Ly6E(hi) neutrophil number was measured (FIG. 4D). Mice with responsive tumors had significantly higher blood levels of Ly6E(hi) neutrophils. After the blood draw, mice were treated with either anti-PD-L1 (B7-H1, BioXCell) or anti-CTLA4 (BioXCell, #BE0164) for two weeks. Tumor growth was assessed twice weekly. Similar to what was observed with anti-PD1 therapy, both anti-PD-L1 therapy (FIG. 4E-F) and anti-CTLA4 therapy (FIG. 4G-H) produced a robust retardation in tumor growth only in the 4T1-M tumors but not 4T1-P tumors. This demonstrates that the mutagenized tumors are sensitive not only to anti-PD1 therapy but to ICI therapy in general. And further identifies the level of Ly6E(hi) neutrophils in peripheral blood as a predictive marker for responsiveness to ICI therapy in general.

### Example 5: Ly6E Neutrophils Overcome Resistance to ICI Therapy

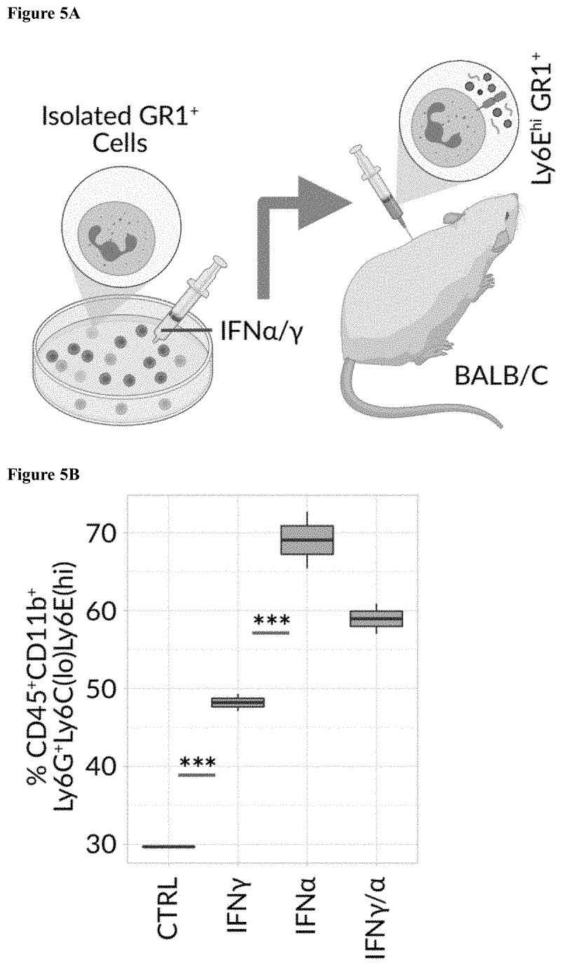

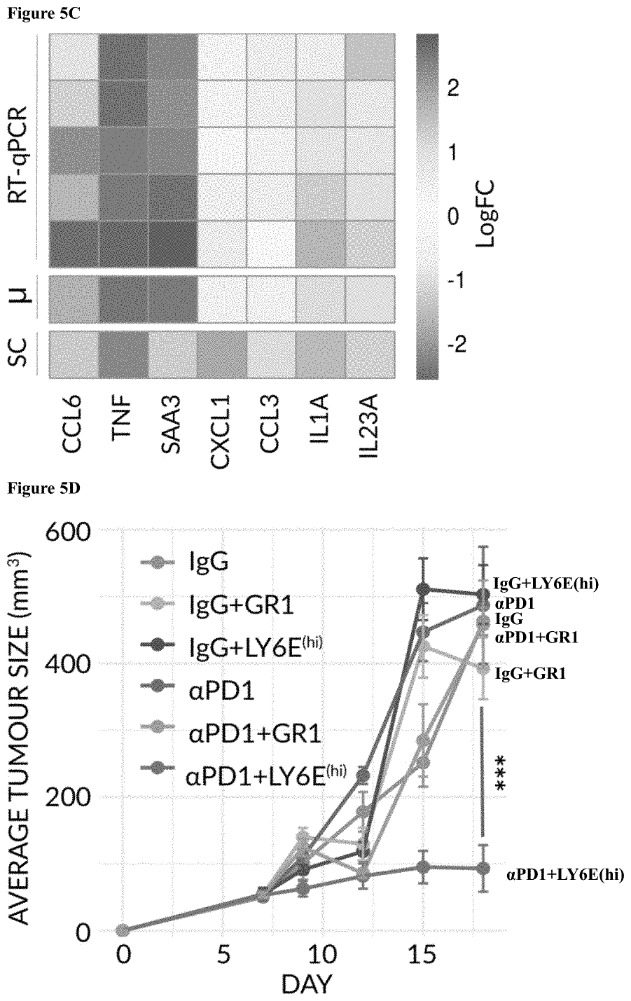

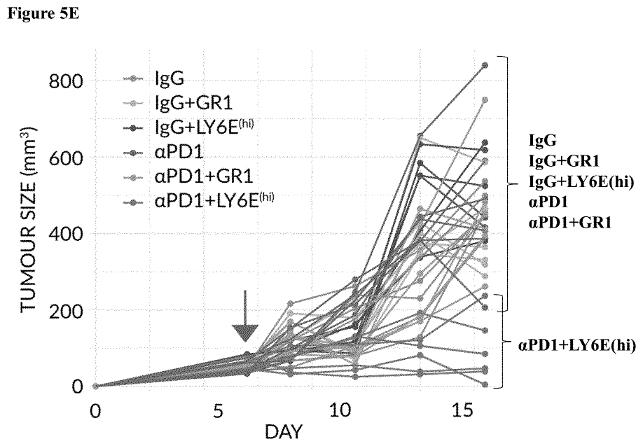

Biomarkers can be surrogate—generated as a byproduct of the main biological mechanism(s)—or they may be functionally involved in the response itself. In order to distinguish between these two possibilities, Ly6E neutrophils were artificially generated in vitro by exposing Gr1+ cells to a cocktail of INFa/g (FIG. 5A) as informed by scRNA-seq analysis (FIG. 3C-D). In order to ensure that the resulting cells resemble the Ly6E(hi) phenotype observed in the scRNA-seq data, the induction of Ly6E at the protein level was analyzed as was the mRNA expression levels (by RT-qPCR) of selected differentially expressed, secreted factors. Firstly, a strong induction of Ly6E on the surface of neutrophils following IFN treatment was observed (FIG. 5B). Secondly, a striking correlation between the log 2FCs of the RT-qPCR (treated vs. untreated) and the scRNA-seq (response vs. non-response) (FIG. 5C) was observed, collectively suggesting these cells are analogous.

Subsequently, their effect, in vivo, on tumors resistant to anti-PD1 was tested by adoptive transfer (FIG. 5A). The administration of Ly6E(hi) neutrophils to mice bearing resistant tumors produced a significant reduction in tumor growth following anti-PD1 therapy but did not display efficacy as a monotherapy (FIG. 5D). Taken together, these results demonstrate that Ly6E(hi) neutrophils not only stratify between responders and non-responders but are also functionally involved in the mechanism of response.

### Example 6: Cells in Human Samples Equivalent to Murine Ly6E(Hi) Neutrophils

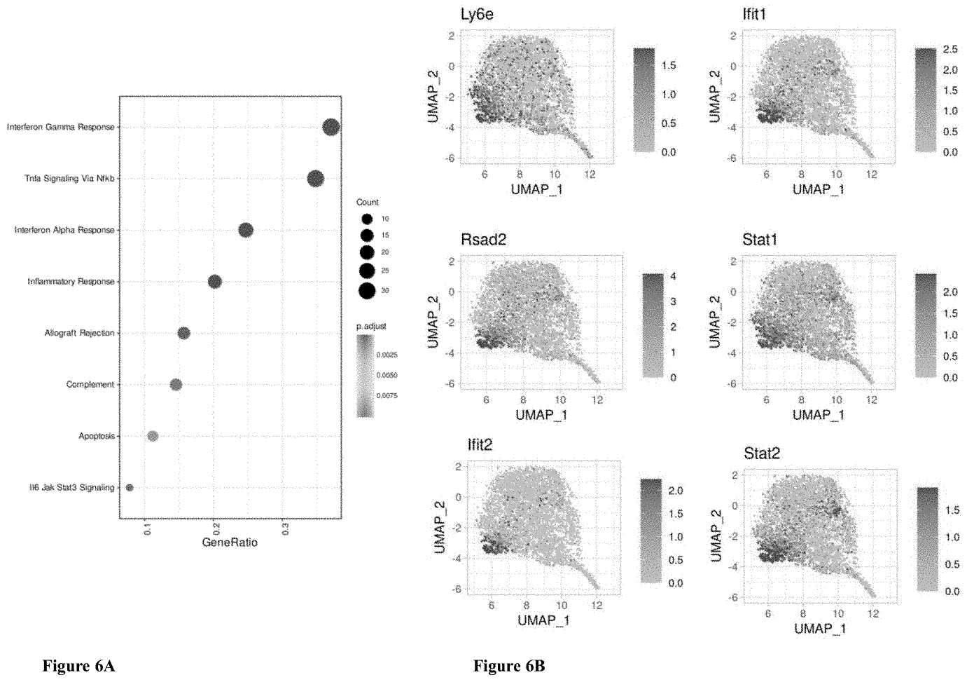

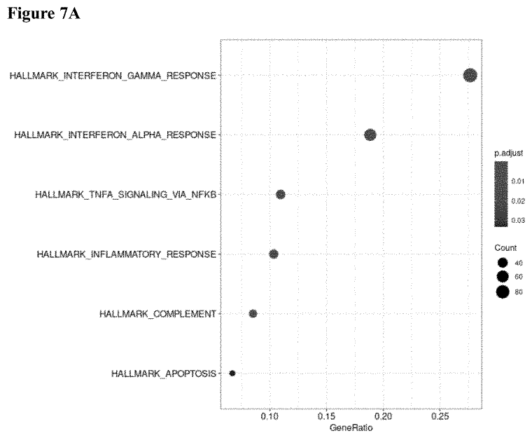

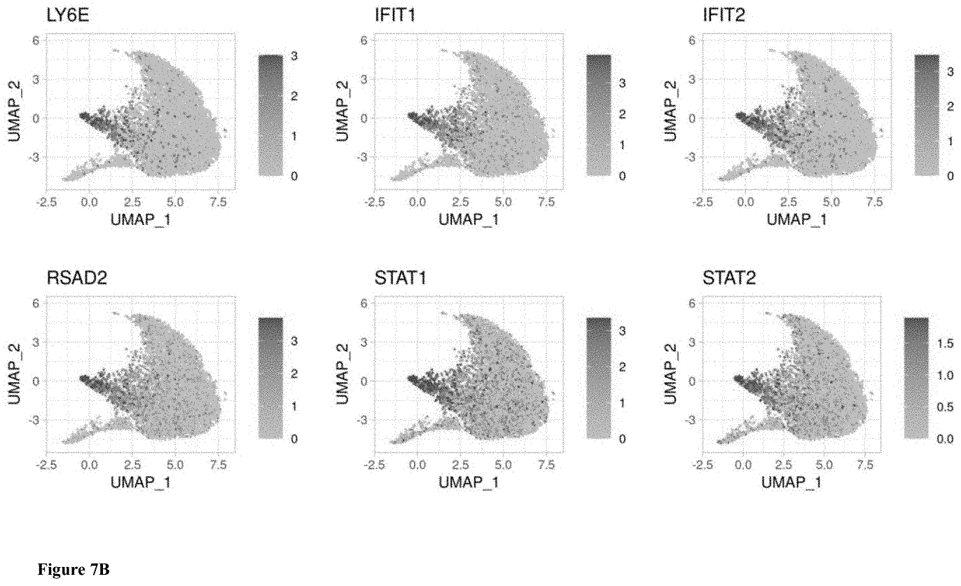

Species specific differences may hinder the ability to translate a biomarker from mouse to human. Specifically, it remained unclear whether Ly6E would be a marker of the same IFN-stimulated cell state in human. Further analysis of our murine scRNA-seq data revealed a distinct gene signature in Ly6E(hi) neutrophils characteristic of response to interferon (IFN)-α/γ (FIG. 6A), including expression of the IFN-inducible genes: LY6E, IFIT1, IFIT2, RSAD2, STAT1, and STAT2 (FIG. 6B). This signature appears to be an IFN-α/γ-stimulated gene signature. The genes unique to Ly6E high neutrophils as compared to all other Ly6G expressing cells are presented in Table 3. This table also contains genes that while not unique to these cells were upregulated in these cells. To assess the feasibility of LY6E(hi) neutrophils as a biomarker for ICI response in humans, this mRNA signature was used to successfully identify equivalent cells within the blood of lung cancer patients (scRNA-seq data, PMID: 30979687) (FIG. 7A-B), demonstrating that these cells are not unique to murine models. In particular, scRNA-data from the blood of 8 NSCLC patients obtained at baseline was analyzed, and the signature was applied to all identifiable neutrophils (FIG. 8A). A cluster of cells highly enriched for the functional signature was observed, it was marked by genes induced by IFN (FIG. 8B). Notably, this cluster displays a high expression of Ly6E, confirming that Ly6E is an appropriate marker of these cells in humans (FIG. 8C).

TABLE 3

| | Genes |
|---|---|
| Uniquely | Ifit1, Cxcl10, Gbp5, Ifi47, Ifit2, Ifih1, Igtp, Slfn8, Gbp3, |
| expressed in | Usp18, Rnf213. Psmb10, Ifi35, Il18bp, Gbp7, Gbp9, Ffar2, |
| mouse Ly6E | Ifit3b, Trim30c, Rgma, Cmpk2, Olfr56, Mitd1, Slfn9, Ntf5, |
| (hi) cells | Trim21, Ifit1bl1, Ly6i, Parp12, Ube2l6 |
| Upregulated | Rsad2, Isg15, Slfn5, Gbp2b, Ly6e, Gbp2, Plac8, Parp14, |
| in mouse | Gbp7, Tnf, Rtp4, Psmb8, Zbp1, Isg20, Ddx60, Oasl2, |
| Ly6E (hi) | Trafd1, Irgm1, Clic4, Bst2, Tap1, Egr3, Stat1, Stat2, |
| cells | Psme2b, Sppl2a, Ddx58, Il23a, Xaf1, Dtx3l, Parp10, |
| | Herc6, Tor3a, Zufsp, Nmi, Trim30a, Trim56, Nlrc5, |
| | Irf7, Parp9, Oas2, Irgm2, Tap2, Tdrd7, Uba7, Il15ra, |
| | Tagap, Gpc3, Daxx |
| Uniquely | IFIT1, ISG15, IFIH1, HERC5, RSAD2, IFI6, MT2A, |
| expressed in | EPSTI1, CMPK2, CMTR1, IFI44L, DHX58, SERTM2, |
| human Ly6E | IFNW1 |
| (hi) cells | |
| Upregulated | SAMD9L, MX1, STAT1, IFIT3, UBE2L6, IFIT5, PARP9, |
| in human | DDX58, BATF2, PARP14, IFIT2, TRIM22, GBP5, |
| Ly6E (hi) | APOL6, IFI16, REC8, OASL, TRIM5, DTX3L, FCGR1B, |
| cells | STAT2, FUT9, SERPING1, GBP1, XAF1, TMEM255B, |
| | ZBBX, PARP12, ETV7, Ly6E |

In order to analyze whether Ly6E(hi) neutrophils in humans mark response to immunotherapy, as in mice, blood samples at baseline were obtained from a new cohort of 34 advanced metastatic NSCLC patients treated with various immunotherapies, and the levels of Ly6E expression in neutrophils were quantified by flow cytometry. The details of the patient cohort are provided in Table 4. Though most of the patients received anti-PD-1 therapy, several received alternative immunotherapies including anti-PD-L1 and anti-CTLA4 therapy. High levels of Ly6E cells (as an absolute value and as a percent of total neutrophils) were correlated with positive outcome as assessed by RECIST at 3 and 6 months, while low levels correlated with poor post-treatment prognosis (FIG. 8D). This correlation was observed regardless of the therapy received. At 6 months a clear stratification was observed. All patients with lower than 30% of neutrophils being Ly6Ehi neutrophils were in a state of progressive disease. All patients with between 40 and 70% Ly6Ehi neutrophils were in a state of stable disease, and all patients with greater than 80% Ly6Ehi neutrophils were found to have a response. This demonstrates that Ly6E is a robust cross-species biomarker.

The same analysis was performed for 16 melanoma patients. These subjects received either anti-PD-1 therapy or anti-CTLA4 therapy. The details of the patient cohort are provided in Table 5. High levels of Ly6E cells (as an absolute value and as a percent of total neutrophils) were correlated with positive outcome (complete or partial response or stable disease) as assessed by response rates based on RECIST at 3 months, while low levels correlated with poor post-treatment prognosis (progressive disease) (FIG. 8E). Ly6E(hi) neutrophils were highly predictive of response for both NSCLC and melanoma as the AUC was 0.94 and 0.98, respectively (FIG. 8F). Surprisingly, Ly6E(hi) neutrophils were much more predictive of ICI response than even PD-L1 staining in the tumor (AUC of 0.94 vs 0.61), a recognized biomarker for PD-1 therapy. Indeed, they were also more predictive than total neutrophil number (AUC of 0.94 vs. 0.75).

TABLE 4
NSCLC cohort

| Diagnosis (lung | | | ORR | ORR | % |
|---|---|---|---|---|---|
| cancer) | Stage | Therapeutic Protocol | 3 m | 6 m | Ly6E(hi) |
| Small cell | IV | Nivo | PD | PD | 23.8 |
| carcinoma | | | | | |
| Adenocarcinoma | III | Cisplatin + Alimta + Pembro | PR | PD | 24.6 |
| Adenocarcinoma | IV | Carbo + Alimta + Pembro | PD | PD | 25 |
| Adenocarcinoma | IV | Carbo + Alimta + Pembro | PR | PD | 25 |
| Adenocarcinoma | IV | Pembro − Lenvima | N/A | PD | 25.3 |
| Adenocarcinoma | IV | Carbo + Alimta + Pembro | PR | PD | 25.9 |
| Adenocarcinoma | IV | Carbo + Alimta + Pembro | PD | PD | 26.1 |
| Adenocarcinoma | IIIA | Durval | N/A | PD | 26.3 |
| SCC | III | Pembro | PR | PD | 27.8 |
| Adenocarcinoma | IV | Nivo | N/A | PD | 28.6 |
| SCC | IV | Pembro | PD | PD | 28.6 |
| SCC | IV | Pembro | SD | PD | 29.3 |
| Adenocarcinoma | III | Carbo + Alimta + Pembro | PR | SD | 42.9 |
| Adenocarcinoma | IIB | Pembro | N/A | SD | 44.7 |
| SCC | II | Pembro | PR | SD | 46 |
| Adenocarcinoma | IV | Carbo + Alimta + Pembro | SD | SD | 47.1 |
| Adenocarcinoma | IV | Carbo + Alimta + Pembro | PR | SD | 50 |
| Adenocarcinoma | IIIA | Durval | N/A | SD | 51.6 |
| Adenocarcinoma | IV | Atezo | PR | SD | 55.6 |
| Adenocarcinoma | IV | Carbo + Alimta + Pembro | PR | SD | 57.8 |
| Adenocarcinoma | IV | Carbo + Alimta + Pembro | PR | SD | 59.4 |
| SCC | IV | Carbo + Taxol + Pembro | SD | SD | 59.4 |
| Adenocarcinoma | IV | Nivo | SD | SD | 60 |
| Adenocarcinoma | IV | Pembro | SD | SD | 62.5 |
| Adenocarcinoma | IV | Atezo | PR | SD | 64.7 |
| NSCLC-NOS | IV | Carbo + Alimta + Pembro | PR | SD | 66 |
| Adenocarcinoma | IV | Carbo + Alimta + Pembro | PR | PR | 84.3 |
| Adenocarcinoma | IV | Carbo + Alimta + Pembro | PR | PR | 84.6 |
| Adenocarcinoma | IV | Carbo − Alimta − Pembro | PR | PR | 87.5 |
| Adenocarcinoma | IV | Carbo + Alimta + Pembro | PR | PR | 88.9 |
| SCC | IV | Carbo + Taxol + Ipi + Nivo | SD | PR | 90 |
| SCC | IV | Pembro | PR | PR | 92 |
| Adenocarcinoma | IV | Carbo + Alimta + Pembro | PR | PR | 93.2 |
| SCC | III | Pembro | PR | PR | 93.7 |

Male (M); Female (F); Squamous cell carcinoma (SCC); Non-small cell lung carcinoma not otherwise specified (NSCLC-NOS); Carboplatin (Carbo); Pembrolizumab (anti-PD1, Pembro); Nivolumab (anti-PD1, Nivo); Atezolizumab (anti-PD-L1, Atezo); Ipilimumab (anti-CTLA-4, Ipi); Durvalumab (anti-PD-L1, Durval); Objective response rate (ORR) is based on response evaluation criteria in solid tumors (RECIST); Partial response (PR); Stable disease (SD), Progressive disease (PD); Information was not acquired (N/A).

TABLE 5
Melanoma

| | | | ORR | % |
|---|---|---|---|---|
| Diagnosis | Stage at samples | Protocol | 3 m | Ly6E(hi) |
| Melanoma | Advanced Disease | Nivolimumab + Ipilimumab + | PD | 21.7 |
| | | Dabrafenib + Trametinib | | |
| Melanoma | Advanced Disease | Ipilimumab | PD | 28 |
| Melanoma | Advanced Disease | Nivolimumab + Ipilimumab + | PD | 31.6 |
| | | Carboplatin + Taxol | | |
| Melanoma | Advanced Disease | Nivolumab + Carboplatin + Avastin | SD | 49.1 |
| Melanoma | Advanced Disease | Nivolimumab + Ipilimumab | PD | 49.8 |
| Melanoma | Advanced Disease | Pembrolizumab | SD | 55.4 |
| Melanoma | Advanced Disease | Pembrolizumab | SD | 59.3 |
| Melanoma | Advanced Disease | Pembrolizumab | CR | 68.4 |
| Melanoma | Advanced Disease | Nivolimumab + Ipilimumab | PR | 69.1 |
| Melanoma | Advanced Disease | Pembrolizumab | PR | 70 |
| Melanoma | Advanced Disease | Nivolimumab + Ipilimumab | PR | 83.8 |
| Melanoma | Advanced Disease | Ipilimumab + Pembrolizumab | SD | 93 |
| Melanoma | Advanced Disease | Nivolumab + Pembrolizumab | PR | 93.6 |
| Melanoma | Advanced Disease | Nivolimumab + Ipilimumab + Taxol | CR | 94.1 |
| Melanoma | Advanced Disease | Pembrolizumab | CR | 95.8 |
| Melanoma | Advanced Disease | Pembrolizumab | CR | 97 |

Pembrolizumab (anti-PD1); Nivolumab (anti-PD1); Ipilimumab (anti-CTLA-4); Dabrafenib (BRAF inhibitor); Trametinib (MEK inhibitor); Carboplatin (Chemotherapy); Taxol (Chemotherapy); Avastin (anti-VEGF-A); Objective response rate (ORR) is based on response evaluation criteria in solid tumors (RECIST); Complete response (CR); Partial response (PR); Stable disease (SD), Progressive disease (PD).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of treating a subject in need thereof with an immunotherapy, the method comprising determining suitability of the subject to be treated with the immunotherapy by receiving a sample from the subject, and measuring Ly6E expression in neutrophils in said sample, wherein the presence in said sample of neutrophils expressing Ly6E above a predetermined threshold indicates said subject is suitable to be treated with said immunotherapy, wherein the method further comprises administering the immunotherapy to the suitable subject or administering the immunotherapy and a pharmaceutical composition of the invention to an unsuitable subject.

2. The method of claim 1, wherein said immunotherapy comprises an immune checkpoint inhibitor (ICI).

3. The method of claim 2, wherein said ICI comprises at least one of anti-PD-1, anti-PD-L1, anti-PD-L2 and anti-CTLA4 immunotherapy.

4. The method of claim 1, wherein said subject suffers from cancer, optionally wherein the cancer is selected from lung cancer, skin cancer, breast cancer, colon cancer, and renal cancer.

5. (canceled)

6. The method of claim 1, wherein said sample is selected from (i) a sample comprising cells; (ii) a cancer sample; (iii) a bodily fluid.

7. (canceled)

8. (canceled)

9. (canceled)

10. The method of claim 1, wherein said sample is acquired from said subject before initiation of administration of said immunotherapy.

11. (canceled)

12. The method of claim 1, wherein said neutrophils are CD45+, HLA-DR−, Lin−, CD11b+, CD33+, CD14−, and CD15+ cells.

13. The method of a claim 1, wherein said neutrophils are myeloid derived suppressor cells (MDSCs).

14. The method of claim 13, wherein said MDSCs are granulocytic MDSCs (G-MDSC).

15. The method of claim 14, wherein said G-MDSC is a polymononuclear (PMN)-MDSC, optionally wherein said PMN-MDSCs are CD45+/CD11b+/Ly6CLow/Ly6G+/Ly6E+ cells.

16. The method of claim 1, wherein said measuring comprises measuring Ly6E surface protein expression or wherein said measuring comprises measuring Ly6E mRNA expression.

17. (canceled)

18. (canceled)

19. (canceled)

20. The method of claim 1, wherein said neutrophils expressing Ly6E above a predetermined threshold make up greater than a predetermined threshold percentage of all neutrophils in said sample.

21. The method of claim 20, wherein said predetermined threshold percentage is 30% of neutrophils, optionally wherein said predetermined threshold percentage is 70% of neutrophils.

22. (canceled)

23. The method of claim 1, wherein said neutrophils expressing Ly6E above a predetermined threshold comprise an mRNA expression profile provided in Table 3.

24. The method of claim 1, wherein said subject is human, and said neutrophils expressing Ly6E above a predetermined threshold is at least one of (i) express at least one of IFIT1, ISG15, IFIH1, HERC5, RSAD2, IFI6, MT2A, EPSTI1, CMPK2, CMTR1, IFI44L, DHX58, SERTM2 and IFNW1.

25. The method of claim 1, herein said subject is human, and said neutrophils expressing Ly6E above a predetermined threshold comprise increased expression of at least one of IFIT3, IFIT1, IFIT2, STAT1, ISG15, STAT2, IFIT5, and IL1B.

26. A pharmaceutical composition comprising a population of neutrophils expressing Ly6E over a predetermined threshold and a pharmaceutically acceptable carrier, excipient or adjuvant.

27. The pharmaceutical composition of claim 26, wherein said composition is formulated for administration to a human subject.

28. The pharmaceutical composition of claim 26, wherein said population of neutrophils expressing Ly6E above a predetermined threshold make up at least 40% of all neutrophils in said composition.

29. (canceled)

30. A method of treating a subject suffering from a disease, the method comprising administering to said subject a pharmaceutical composition of claim 26 and an immunotherapy, thereby treating said subject.

31.-37. (canceled)
